(19) United States
(12) Patent Application Publication
(10) Pub. No.: US 2021/0308111 A1
(43) Pub. Date: Oct. 7, 2021

(54) Clinical Methods And Pharmaceutical Compositions Employing AMPA Receptor Antagonists To Treat Glioblastoma And Other Cancers

(71) Applicant: WITH GREAT POWER, LLC, Glen Rock, NJ (US)

(72) Inventor: Daniel Pierce RADIN, Glen Rock, NJ (US)

(21) Appl. No.: 17/261,857

(22) PCT Filed: Jul. 25, 2019
(86) PCT No.: PCT/US2019/043525

§ 371 Date: Jan. 20, 2021

Related U.S. Application Data
(60) Provisional application No. 62/703,951, filed on Jul. 27, 2018.

(51) Int. Cl. A61K 31/444; A61K 45/06; A61P 35/00; A61K 9/00

(52) U.S. Cl. CPC A61K 31/444; A61K 9/0053; A61P 35/00; A61K 45/06

(57) ABSTRACT

Disclosed herein are compositions and methods employing AMPA Receptor (AMPR) antagonist compounds to treat AMPAR positive cancers in mammalian subjects. In certain detailed embodiments the AMPAR antagonist is a Perampanel compound, effective to mediate potent oncolytic effects to prevent or reduce the severity or recurrence of a variety of cancer forms, including central nervous system (CNS) cancers.

### TECHNICAL FIELD

The invention relates to drugs and clinical methods for treating cancer in mammalian subjects. More specifically the invention relates to treating glioblastoma and other cancers that are positive for expression of AMPA-receptors.

### CROSS-REFERENCE TO RELATED APPLICATIONS

This application is related to and claims the priority benefit of prior U.S. Provisional Patent Application No. 62/703,952, filed Jul. 27, 2018, and prior U.S. Provisional Patent Application No. 62/796,032, filed Jan. 23, 2019, each incorporated herein by reference in its entirety for all purposes.

### BACKGROUND OF THE INVENTION

Cancer remains a principal mortality risk in human populations, with available drugs and treatment methods falling well short of goals to effectively treat and manage all forms of cancer in diverse patients. Today cancer persists as the second leading cause of death in the United States and in other developed nations. The US National Cancer Institute (NCI) reported 8.2 million cancer-related deaths worldwide and 14.1 million new cases diagnosed in 2012. New cancer diagnoses are projected to rise globally to roughly 24 million by 2030. According to current NCI statistics, an estimated 1,735,350 new cases of cancer will have been diagnosed and 609,640 cancer deaths tolled in the US for the year 2018.

The economic burdens of diagnosing and treating cancer on healthcare systems around the world are enormous, with estimated national expenses for cancer care in the United States in 2017 approaching $150 billion. Cancer health costs will continue to rise as mean population age and cancer prevalence increase, and more expensive treatments are adopted as standards of care.

Conventional treatments for cancer typically involve a combination of surgery, chemotherapy, radiation and hormonal therapy. Each of these treatment modalities imposes significant morbidity and added risks, for example adverse metabolic and reproductive impacts on healthy cells, immunosuppression and attendant increased risks of infection, and many other adverse health conditions that attend the rigors and insults of conventional cancer therapy.

Despite considerable advances in detection and treatment of cancer over the past several decades, conventional treatments like surgery, chemotherapy and radiation often achieve only modest improvements in survival, while imposing significant adverse impacts on quality of life—raising questions about cost-effectiveness and overall clinical benefits of such treatments.

In view of the foregoing there persists a dire and compelling need in the medical arts for alternative tools and methods to prevent, treat and clinically manage cancer.

Glioblastoma (GBM) is a high-grade cancer of the central nervous system (CNS) characterized by a highly invasive and treatment resistant phenotype. Patients almost always relapse after an either initially successful surgical resection with or without chemo- and radiotherapy (Ishiuchi et al. 2007). GBM tumors often exhibit resistance to chemotherapy and/or radiotherapy, which resistance may be acquired during a course of treatment (Ishiuchi et al. 2007).

Temozolomide (TMZ) is a DNA methylating agent that is the current standard of care drug for treating GBM. TMZ mediates anti-cancer effects through genotoxic activity, and is effective in GBM treatment due in part to the drug's ability to bypass the blood-brain barrier (BBB) (Prasad et al. 2011). Unfortunately, TMZ shows limited efficacy for long-term treatment of GBM, and many patients appear to be refractory to TMZ treatment.

While considerable research has been attempted to elucidate the pathophysiology of GBM, a vast majority of drugs tested against GBM are unable to pass the BBB to yield sufficient drug levels in the brain to mediate anti-cancer effects (Liu et al. 2015).

Previous reports have suggested a role for glutamate in the proliferation and migration of glioma cells, in a manner similar to glutamate function in neuronal development (Rzeski et al. 2001; Ishiuchi et al. 2002). Tumors of the CNS that release glutamate may cause cytotoxicity and cell death in neighboring neurons, which is postulated to facilitate cancer invasion into neighboring tissues (Takano et al. 2001). Glutamate positive tumors may also grow at an enhanced rate, potentially implicating glutamate signaling as an important mechanism in the etiology of GBM (Takano et al. 2001).

Among many types of known glutamate receptors (GRs), the AMPA-type glutamate receptor (AMPAR) may be overexpressed in certain ty pes of cancer, including some forms of CNS cancers (Liu et al. 2015) and non-CNS cancers (Stepulak et al. 2007; Herner et al. 2011; Romeling et al. 2014; Hu et al. 2014). AMPAR activation may be linked to increased cancer cell invasiveness (Piao et al. 2008), proliferation, and/or activation of the PI3K/Akt/mTOR signaling axis (Ishiuchi et al. 2007). The PI3K/Akt/mTOR signaling axis has been implicated in chemotherapy resistance in GBM, and available drugs reported to disrupt this pathway, such as rapamycin, do not penetrate the BBB. GBM cancer stem cells (suspected to be capable of re-establishing tumors alter ablation with surgery, chemotherapy or radiotherapy) may express strikingly high amounts of functional AMPARs (Oh et al. 2012).

In view of the foregoing, a compelling need exists in the art for new drugs and therapeutic methods for treating cancer. Related needs are unmet for new drugs for treating refractor) or treatment-resistant forms of cancer, including cancers of the CNS. For CNS cancers, particularly brain cancers such as GBM, there is a particularly urgent need for anti-cancer drugs capable of transiting the BBB to yield effective drug concentrations within protected CNS compartments, most notably within the brain.

### SUMMARY OF EXEMPLARY EMBODIMENTS OF THE INVENTION

The instant invention satisfies the foregoing needs and fulfills additional objects and advantages by providing novel AMPAR antagonist drugs effective to treat AMPAR positive cancers and AMPAR dependent cancers, including AMPAR positive and AMPAR dependent cancers of the central nervous system (CNS). In exemplary embodiments the invention provides compositions and methods employing a novel anti-cancer drug, Perampanel (PMP) [2-(2-oxo-1-phenyl-5-pyridin-2-yl-1,2-dihydropyridin-3-yl) benzonitrile], heretofore reported for limited clinical use as an antispasmodic drug.

AMPA receptor antagonists have been investigated for antiseizure activity both preclinically and clinically, with mixed success. The prototypical competitive AMPA receptor antagonist 2,3-dihydroxy-6-nitro-7-sulfamoyl-benzo[f] quinoxaline (NBQX) showed activity in maximal electroshock (MES) and pentylenetetrazole (PTZ)-induced seizure models (Yamaguchi et al., 1993), but has poor solubility, resulting in precipitation in the kidney at therapeutic plasma levels. Derivatives of NBQX with polar constituents have shown improved solubility, but these molecules exhibit reduced blood-brain barrier (BBB) penetration (Weiser, 2005). Prototypical noncompetitive AMPA receptor antagonists, such as 2,3-benzodiazepine-type compounds, have shown weak in vitro efficacy compared with competitive antagonists (Weiser, 2005). Talampanel, a recently developed noncompetitive AMPA receptor antagonist, has been evaluated in a number of clinical trials (Howes & Bell, 2007), but has a relatively short hall-life militating against its potential clinical utility (Langan et al., 2003). More recently. Steinhoff et al., 2013, reported beneficial activity of Peramplanel, a noncompetitive, selective AMPA receptor antagonist, as an antiepileptic drug undergoing clinical study for refractory partial-onset seizures.

According to the surprising discoveries herein, peramplanel (PMP) has now been identified to possess novel and potent anti-cancer activity. Within the compositions and methods of the invention. PMP, along with its active analogs and derivatives, and other selected AMPAR antagonists disclosed herein, potently inhibit AMPAR positive and AMPAR dependent cancers, including CNS cancers such as GBM.

The invention provides novel compositions and methods for treating cancer using AMPAR antagonist compounds such as PMP to reduce or prevent the occurrence, remission, growth, severity and/or one or more adverse symptom(s) of AMPA-receptor positive cancers in mammalian subjects, including humans. In illustrative embodiments, the AMPAR antagonist comprises a PMP compound (including anti-cancer effective chemical analogs, derivatives, conjugates, solid crystalline forms, solvates and/or different salt forms of a PMP compound), which is clinically effective as an anti-cancer agent to treat or prevent cancer in mammalian subjects. In exemplary embodiments. PMP is administered to a human patient presenting with an AMPAR positive cancer condition in a delivery mode, formulation and dosage sufficient to alleviate one or more symptoms of the targeted cancer condition in the patient.

In certain embodiments the PMP compound is perampanel [2-(2-oxo-1-phenyl-5-pyridin-2-yl-1,2-dihydropyridin-3-yl) benzonitrile] formulated in a biologically acceptable composition for administration to a human subject.

In related embodiments, novel clinical methods are provided herein employing a peramplanel or related compound administered to a mammalian subject, wherein the peramplanel compound exerts oncolytic effects against a targeted cancer cell or tumor sufficient to kill a targeted cell or tumor, reduce site of a tumor, impair tumor growth, prevent or reduce cancer invasiveness, reduce or delay cancer recurrence, and/or alleviate one or more symptoms associated with the treated cancer condition.

In more detailed embodiments, peramplanel and related compounds are employed in effective anti-cancer methods for treating glioblastoma (GBM). The peramplanel compound is administered to a mammalian subject with current or prior diagnosis of GBM in a dosage form, amount and regimen sufficient to prevent or reduce the occurrence, severity, recurrence and/or related symptoms of GBM in the subject. In related embodiments, pharmaceutical compositions and delivery methods are provided that yield surprisingly high therapeutic concentrations of the peramplanel compound in a CNS compartment of the subject, e.g., in the brain, yielding potent anti-CNS-cancer therapeutic effects.

In other detailed embodiments, a peramplanel compound is administered with a secondary therapeutic agent in combinatorial formulations or coordinate treatment methods to yield desired therapeutic advantages. In exemplary embodiments, a peramplanel compound is coordinately administered with a second anti-cancer drug to treat cancer, whereby anti-cancer efficacy is enhanced and/or adverse side effects are reduced. In one illustrative embodiment, peramplanel is coordinately administered with temozolomide (TMZ) to treat GBM, pancreatic cancer, or another form of cancer. In another embodiment, peramplanel is coordinately administered with cisplatin to treat an AMPAR positive cancer, for example an AMPAR positive pancreatic cancer. In other exemplary embodiments, peramplanel is coordinately administered with hydroxyurea to treat an AMPAR positive cancer. In other embodiments, peramplanel is coordinately administered with Carmustine (BCNU) to treat an AMPAR positive cancer.

In related treatment methods, a peramplanel compound is coordinately administered before or after a conventional cancer treatment, for example surgery, chemotherapy or radiation treatment, with or without a secondary anti-cancer agent or other secondary therapeutic drug.

In other detailed aspects of the invention, methods and compositions are provided employing a peramplanel compound to reduce oncogenic activity by disrupting a glutamate-induced cancer potentiation process (e.g., glutamate-stimulated cancer cell proliferation, tumor growth, cancer invasion or other cancer-potentiation activity).

In yet additional embodiments of the invention. AMPAR antagonist compounds are employed in novel clinical methods and compositions to treat lung cancers, breast cancers, pancreatic cancers, liver cancers, colorectal cancers and other forms and symptoms of cancer conditions in human subjects.

### BRIEF DESCRIPTION OF THE DRAWINGS

FIG. 1 is a related graph series documenting the effects of PMP on T98G GBM cell viability. Dose-response curve of A) PMP on cell viability B) Interactions with PMP and TMZ. Data are presented as mean+/−SEM of 2 or more independent experiments performed in quadruplicate. ANOVA. P<0.001 for PMP suggesting a significant dose-dependent response. *p<0.05. **p<0.01, t-test, compared to vehicle-treated control or singular drug treatment. +p<0.05, king's synergy test, demonstrating significant synergistic interaction.

FIG. 2 is a related graph series documenting the effects of PMP on Panel cell viability. Dose-response curve of: A) PMP on cell viability; B) Interactions with PMP and 3 uM cisplatin; C) Effects of glutamate on panel cell viability; and D) Interactions between glutamate and PMP. Data are presented as mean+/−SEM of 2 or more independent experiments performed in quadruplicate. ANOVA. P<0.001 for PMP and glutamate, indicating a significant dose-dependent response. *p<0.05. **p<0.01, t-test, compared to vehicle-treated control or singular drug treatment. +p<0.05, ++p<0.01, king's synergy test, demonstrating significant synergistic interaction. *p<0.01, t-test, compared to viability of glutamate- or PMP-treated cells alone.

FIG. 3 is a flow chart illustrating seven candidate anti-cancer chemical derivatives (D1-D7) of peramplanel (PMP), herein PMP is modified according to known methods of conventional rational design chemistry, to yield new candidate compounds for testing to determine anti-cancer activity and other beneficial properties.

### DETAILED DESCRIPTION OF EXEMPLARY EMBODIMENTS OF THE INVENTION

The invention provides AMPAR antagonist, compounds exemplified by peramplanel (PMP), shown to be surprisingly effective in treating cancers, including CNS cancers, in mammalian subjects. Among the discoveries presented here, peramplanel is shown to exert potent, direct oncolytic effects against cancer cells in assays accepted to predict clinical anti-cancer activity in human subjects. More specifically the examples below show that PMP potently disables viability of CNS and non-CNS cancers, as demonstrated by direct oncolytic effects against glioblastoma (GBM) and pancreatic cancer cells. Related studies further evince that peramplanel exerts surprisingly potent, additive or synergistic anti-cancer effects in coordinate use with other chemotherapeutic drugs.

The clinical methods and pharmaceutical compositions and formulations of the invention provide novel tools to treat, prevent and clinically manage a wide range of cancers in mammalian subjects, including humans. Any type and form of cancer occurring in humans and veterinary subjects may be amenable to treatment according to the teachings herein, including but not limited to: central nervous system (CNS) cancers including various forms of brain cancer; lung cancer; prostate cancer; breast cancer; skin cancers, for example melanoma; liver cancer; thyroid cancer; esophageal cancer; sarcomas; colon and rectal cancers; bladder cancer; gall bladder cancer; stomach cancer; renal cancer; ovarian cancer; uterine cancer; cervical cancer; non-Hodgkin's lymphoma; acute myelogenous leukemia (AML); acute lymphocytic leukemia; chronic lymphocytic leukemia (CLL); myeloma; mesothelioma; pancreatic cancer, Hodgkin's disease; testicular cancer; Waldenstrom's disease; head/neck cancer; cancer of the tongue, viral-induced malignancies (e.g., cancers induced by SV₄₀ virus), and other candidate types and forms of cancers that will be apparent to skilled artisans.

Subjects amenable to treatment may have cancer of any stage of development and etiology, including, but not limited to, cancers marked by rapid increases in cellular/histological abnormalities and/or elevated tumor marker expression in biopsies or blood samples, rapid tumor proliferation and/or growth, metastasis, among other disease progression indicators, up to and including stage III and stage IV cancers, even refractory stage III and IV shown to be “treatment resistant cancers” (e.g., to effectively subjects with cancers, such as glioblastoma, persisting or relapsing after ineffective, conventional anti-cancer treatment(s) (e.g., surgery, radiation and/or chemotherapy)). In exemplary embodiments of the invention an effective AMPAR antagonist drug such as PMP effectively prevents or treats (i.e., reduces the severity, progression and/or adverse side effects of) cancer in treatment resistant subjects, defined as subjects presenting after one or more rounds of conventional oncotherapy (e.g., chemotherapy, radiation, surgery and/or hormonal therapy), with actively progressing or unstable metastatic disease. In other embodiments the compositions and methods of the invention are useful for treating other “refractory” patients who may not otherwise tolerate or be fit for conventional cancer treatments such as chemotherapy.

Certain cancer types and disease conditions are contemplated herein to be particularly amenable to treatment using the AMPAR antagonist drugs and methods of the invention. In certain embodiments, the AMPAR antagonist drugs and methods of the invention are particularly effective against “AMPAR dependent” cancers. As used herein the term “AMPAR dependent refers to cancers that distinctly overexpress AMPA receptors, or whose appearance, growth, and/or disease progression may otherwise be determined to be AMPAR-dependent. In more detailed aspects, “AMPAR-dependent” cancers are not limited to cancers %% hose occurrence, persistence or progression require abnormally elevated AMPAR receptor expression or activity, indeed they may include cancers with normal or even subnormal expression of AMPARs, which through disease-associated changes in AMPAR structure or function, or any other disease-associated change affecting AMPAR metabolism or pathology, are particularly susceptible to AMPA receptor interference or blockade using PMP or other candidate AMPAR drugs of the invention.

In this context, the use of AMPAR antagonist drugs exemplified by PMP, according to the teachings herein, effectively treats or prevents a wide range of cancers contemplated to represent “AMPAR-dependent cancers”, including but not limited to brain cancer, breast cancer, colorectal cancer, hepatocellular cancer, leukemia, melanoma, lung cancer, pancreatic cancer, renal cancer, and other candidate cancer types or cases determined to be clinically susceptible to AMPAR interference or blockade by PMP or another useful AMPAR antagonist.

Each of the anti-cancer methods of the invention involves administration of a suitable, effective dosage amount of PMP or another useful AMPAR antagonist to a subject. Typically, an effective amount will comprise an amount of the active compound (e.g., PMP) which is therapeutically effective, in a single or multiple unit dosage form, over a specified period of therapeutic intervention, to measurably alleviate the targeted cancer condition. Within exemplary embodiments, PMP is used as the sole or primary active drug. In other embodiments, an intermediary or precursor compound to PMP, or a rationally-designed analog or derivative of PMP (i.e., a related compound having close structural and functional similarity to PMP) is employed. The PMP or other effective AMPAR antagonist is typically formulated in a pharmaceutical composition with one or more pharmaceutically acceptable carriers, excipients, vehicles, emulsifiers, stabilizers, preservatives, buffers, and/or other additives that may enhance stability, delivery, absorption, half-life, efficacy, pharmacokinetics, and/or pharmacodynamics, reduce adverse side effects, or provide other advantages for pharmaceutical use.

Anti-cancer effective dosage amounts of PMP and other effective, anti-cancer AMPAR antagonists of the invention will be readily determined by those of ordinary skill in the art, depending on clinical and patient-specific factors. Suitable effective unit dosage amounts of the active compounds for administration to mammalian subjects, including humans, may range from a minimum daily dose of 1-2 mg up to a maximum prospective dose between about 200-500 or 300-1,000 mg/day, or greater. In certain embodiments, the anti-cancer effective dose is between about 2 mg-200 mg/day, in other embodiments between about 20-400 mg/day, 50-500 mg/day, 200-600 mg/day, or another anti-cancer effective dose or dosage range that can be adjusted based on patient specific factors to optimize efficacy and minimize adverse side effects. The PMP or other AMPAR antagonist may be administered in a single dose, or in the form of a multiple periodic dosing protocol, for example in a dosing regimen comprising from 1 to 5, or 2-3 doses administered per day, per week, or per month.

The amount, timing and mode of delivery of the anti-cancer compositions of the invention will be routinely adjusted on an individual basis, depending on such factors as patient weight, age, gender, and condition of the individual, the acuteness of the subject's disease and severity symptoms, whether the administration is prophylactic or therapeutic, prior treatment history (including e.g., any prior history and responsiveness to chemotherapy or other cancer treatment treatment) and on the basis of other factors known to effect drug delivery, absorption, pharmacokinetics and efficacy. An effective dose or multi-dose treatment regimen for the instant AMPAR antagonist formulations will ordinarily be selected to approximate a minimal dosing regimen necessary and sufficient to substantially prevent or alleviate the cancer condition, and/or to substantially prevent or alleviate one or more symptoms associated with that condition.

A dosage and administration protocol will often include repeated dosing therapy over a course of several days or even one or more weeks, up to several months, or even a year or more. An effective treatment regime may also involve prophylactic dosage administered on a daily or multi-dose per day basis lasting over a course of days, weeks, months or even a year or more.

Various assays and pre-clinical and clinical model systems can be readily employed to determine therapeutic effectiveness of the anti-cancer compositions and methods invention. For example, these may detect/monitor a decrease in overt symptoms, such as pain (e.g., as measured using an of a variety of pain scales including, but not limited to, the Visual Analog Scale. McGill Pain Questionnaire. Descriptor Differential Scale. Faces Pain Scale. Verbal Rating Scale. Simple Descriptive Pain Scale. Numerical Pain Scale (NPS), or Dolorimeter Pain Index). More detailed detection/monitoring may document, for example, a decrease in circulating tumor cells (CTCs), reduction in tumor size, collapse or disappearance of tumors, softening of tumors, liquefaction of tumors, or a decrease in cytological or histochemical cancer markers, among many other conventional diagnostic indicia of cancer disease stasis, progression and/or remission.

Effectiveness of the anti-cancer methods and compositions of the invention are generally demonstrated by a decrease in incidence, severity and/or associated symptoms of cancer, which will typically involve a decrease of 5%, 10%, 25%, 30%, 50%, 75%, 90% or more in comparison to incidence/levels of the same diagnosed indicator/state, or attendant symptom(s) in suitable control subjects (or compared to known baseline or median data for like, treated or untreated subjects). For example, PMP-treated cancer patients will often exhibit a decrease in number or size of targeted tumors, a decrease in circulating tumor cells (CTCs) or Cancer Stem Cells (CSCs) in successive blood assays, and/or a decrease in one or more tumor-associated cytological, histochemical or blood markers, during a course of treatment, of from 25%-30%, 50%, 75% or higher, 90% and up to total absence of the disease indicator(s) to a limit of detectability associated with the employed assay(s). Monitoring for effective cancer prevention and treatment of the invention can employ any of a vast array conventional detection and monitoring tools and indicia, as will be apparent to those skilled in the art. For example, CTC monitoring using blood samples of patients can utilize flow cytometry, immunobead capture, fluorescence microscopy, standard and density centrifugation, cell culturing, and immunocytochemistry. Similarly, tumor monitoring can employ x-ray. MRI, CT or PET scans, among other methods and tools. For economy these and other routine, well-known cancer disease detection and monitoring technologies will not be reiterated here.

As noted above, exemplary embodiments of the invention employ peramplanel (PMP) as an anti-cancer effective AMPAR antagonist compound. Perampanel is structurally distinct from other AMPAR antagonists, which as a group show a great deal of structural diversity (for example, as illustrated in Table I comparing the structure of PMP to two other AMPAR antagonists, Telampanel and NBQX.

```
TABLE 1
Diverse Chemical Structure of AMPAR Antagonists
  Perampanel
  NBQX
  Talampanel
```

According to the discoveries herein, PMP potently reduces or prevents the occurrence, remission, growth and/or severity of targeted cancers in mammalian subjects, including humans. In certain embodiments. PMP is effective to prevent or treat (including to reduce one or more adverse symptom(s) of), an AM PAR positive cancer in a human cell population, tissue, organ or whole patient. For clinical use, effective anti-cancer compositions may comprise a prototypical PMP compound [2-(2-oxo-1-phenyl-5-pyridin-2-yl-1,2-dihydropyridin-3-yl) benzonitrile], or any effective prodrug, metabolite, analog, derivative, conjugate, solid crystalline form, solvate and/or advantageous salt form of PMP shown to be clinically effective as an anti-cancer agent.

In view of the disclosed potent anti-cancer effects of peramplanel (PMP), the invention further provides various chemical analogs and derivatives of PMP that actively treat or prevent cancer, and in certain cases provide additional clinical advantages, for example improved solubility, enhanced blood-brain barrier penetration, prolonged half-life, increased AMPAR antagonist activity, among other functional improvements.

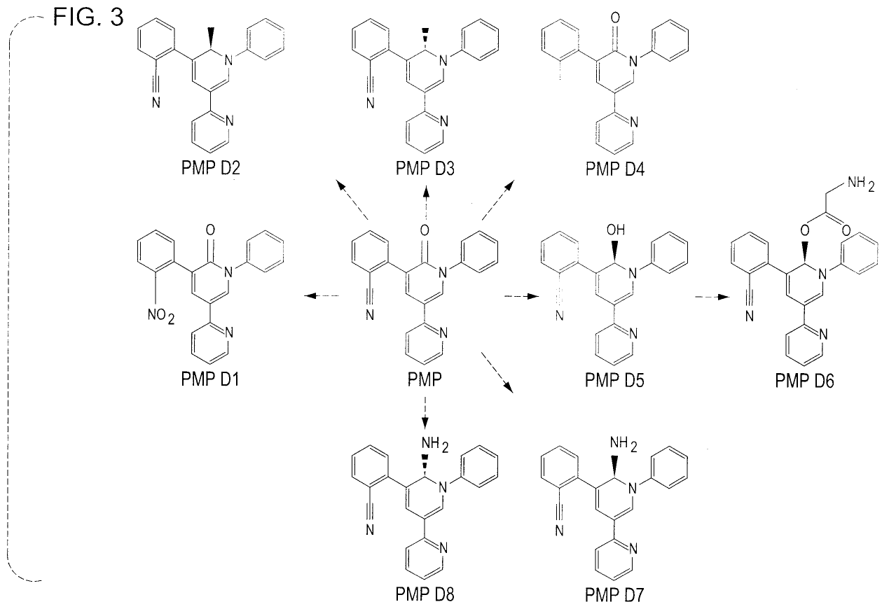

FIG. 3 provides a flow chart illustrating seven candidate anti-cancer chemical derivatives (D1-D7) of peramplanel (PMP) contemplated for anti-cancer testing within the clinical methods herein. The illustrated compounds, D1-D7 can be readily produced, along with many additional PMP analogs and derivatives, employing known methods of conventional rational design chemistry. Such routine design and synthesis efforts will yield a diverse array of new candidate compounds for testing within the methods of the invention, to determine anti-cancer activity and other beneficial properties. In general terms, each of the available R groups identified within or attached to each of the aromatic rings of PMP can be altered (e.g., by chemical deletion, substitution or addition) to yield new candidate PMP derivative drugs as described. The exemplary derivatives shown in FIG. 3—PMP D1 may be designed, tested and selected to have increased solubility, improved half-life, better delivery or penetration to desired targets or compartments (e.g., to deliver an effective dose within a tumor mass, to transit the blood brain barrier (BBB) in anti-cancer effective amounts, to survive first pass metabolism and transport to a target organ in effective plasma concentration, etc.). According to the illustrative embodiments here, PMP derivatives D2, D3 and D4 are designed to have more hydrophilic character, whereby they will have improved BBB penetration and accumulate to an effective concentration greater in the CNS to mediate clinical benefits of treating and/or preventing CNS related cancers such as GBM. PMP D5 is designed to provide increased solubility for improved drug delivery, bioavailability and clinical efficacy. PMP D6 is an exemplar prodrug (a glycine ester) of PMP D5, which will be rapidly hydrolyzed into PMP D5 in the blood by plasma glycine esterases, thus providing the contemplated prodrug benefits in addition to increased solubility. PMP D7 and D8 are likewise more hydrophilic derivatives of PMP which will penetrate the BBB and accumulate in effective concentrations for anti-cancer drug effects in the CNS.

In other exemplary PMP derivative and analog design, each R group may be modified to a same, or different, derivative or analog R group identity. Rational design chemical alterations to PMP can include alterations wherein an original PMP R group is altered to a new structural identity selected from, for example: a substituted or unsubstituted lower hydrocarbon including an alkyl, alkenyl, alkanoyl, alkynyl, aryl, aroyl, aralkyl, alkylamino, aryloxy, hydrogen, carboxyl, nitro, thioalkoxy, thioaryloxy, thiol, cycloalkenyl cycloalkyl, heterocycloalkyl, heteroaryl, aralkyl, amino acid, peptide, dye, fluorophore, carbohydrate or polypeptide: a hydrogen, hydroxyl, sulfhydryl, fluorine, methyl, ethyl, propyl, benzyl, 2-bromovinyl amino, hydroxymethyl, methoxy, halogen, pseudohalogen, cyano, carboxyl, nitro, thioalkoxy, thioaryloxy, or thiol; a substituted or unsubstituted lower hydrocarbon containing 1 to 20 carbons such as alkoxycarbonyl, alkoxycarbonylamino, amino, amino acid, aminocarbonyl, aminocarbonyloxy, aralkyl, aryloxy, carboxyl, cycloalkenyl, alkyl, cycloalkyl, heterocycloalkyl, aryl, heteroaryl, aralkyl, amino acid, peptide, dye, fluorophore, carbohydrate or polypeptide: a heteroatom such as oxygen, sulfur or nitrogen; and/or an integral member of a new 5, or 6, member exocyclic ring structure, among other alterations where feasible to yield a viable test candidate derivative. In more detailed embodiments, one or more R group(s) of PMP can be modified to a hydrogen, hydroxyl, sulfhydryl, benzyl, 2-bromovinyl amino, hydroxymethyl, methoxy, halogen, pseudohalogen, cyano, carboxyl, nitro, thioalkyl, thioaryl, thiol, substituted or unsubstituted hydrocarbons containing 1 to 20 carbons, alkoxycarbonyl, alkoxycarbonylamino, amino, amino acid, aminocarbonyl, aminocarbonyloxy, aryloxy, carboxyl, cycloalkenyl, substituted or unsubstituted cycloalkyl, substituted or unsubstituted heterocycloalkyl, substituted or unsubstituted aryl, substituted or unsubstituted heteroaryl, substituted or unsubstituted aralkyl, peptidyl, dye, fluorophore, carbohydrate or polypeptidyl, azido, nitrile, substituted benzoyl or hydroxyl substituted with substituted or unsubstituted hydrocarbon containing 1 to 20 carbons, and/or an alkanoyl of a main chain of 1 to 20 carbon atoms, among many other contemplated derivative changes obtainable and testable according to the teachings herein without undue experimentation.

Within additional aspects of the invention, combinatorial formulations and coordinate treatment methods are provided that employ an effective amount of PMP or another anti-cancer effective AMPAR antagonist compound or composition, and one or more secondary or adjunctive agent(s) combinatorially formulated or coordinately administered with the AMPAR antagonist compound or composition to yield an enhanced anti-cancer composition or method. Exemplary combinatorial formulations and coordinate treatment methods in this context employ a PMP compound in combination with the one or more secondary anti-cancer, anti-viral, and/or immune-stimulatory effective agents or drugs.

Exemplary combinatorial formulations and coordinate treatment methods of the invention employ PMP or another anti-cancer effective AMPAR antagonist compound or composition in combination with one or more secondary or adjunctive anti-cancer effective agents, for example one or more chemotherapeutic agents. Employing general terminology for “chemotherapeutic drugs and adjunctive anti-cancer therapies”, these secondary agents/therapies for use within the invention may include any anti-cancer or anti-proliferative agent, agents that destroy or “reprogram” cancer cells, agents that destroy blood vessels associated with neoplasms or hyperproliferative conditions, and other classes of drugs harmful to neoplastic cellular targets. In this regard, useful chemotherapeutics and adjunctive therapies for use within the invention include, but are not limited to:
(1) Tubulin depolymerizing agents like toxoids;
(2) DNA damaging agents and agents that inhibit DNA synthesis;
(3) Anti-metabolics;
(4) Anti-angiogenic agents and vascular disrupting agents (VDAs);
(5) Anti-cancer antibodies;
(6) Endocrine cancer therapies;
(7) Immuno-modulators;
(8) Histone deacetylase inhibitors;
(9) Inhibitors of signal transduction;
(10) Inhibitors of heat shock proteins;
(11) Retinoids;
(12) Growth Factors and Modulators of growth factor receptors;
(13) Anti-mitotic compounds;
(14) Anti-inflammatory agents such as COX inhibitors; and
(15) Cell cycle regulators (eg. check point regulators and telomerase inhibitors).

In related embodiments, coordinate anti-cancer treatment methods of the invention can include coordinate administration of one or more anti-cancer AMPAR antagonist compounds with a secondary anti-cancer agent selected from azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, docetaxel, palifermin, panitumumab, pegaspargase, pemetrexed disodium, rituximab, soratenib tosylate, sunitinib malate, tamoxifen citrate, targretin, temozolomide, thalidomide, and/or topotecan hydrochloride. Additional contemplated secondary anti-cancer effective agents in this context include, but are not limited to, interleukin-2, interferon α, filgrasten, G-CSF, epoetin alfa, erythropoietin, IL-1, oprelvekin, trastuzumab, vorinostat, antibiotics, coenzyme q, palladium lipoic complexes including, for example, poly-MVA®, antineoplastins, cartilage, hydrazine sulfate; milk thistle, electrolytes such as calcium carbonate, magnesium carbonate, sodium bicarbonate, and potassium bicarbonate; oxidizing agents, including, but not limited to, cesium chloride, potassium chloride, potassium orotate and potassium aspartate; immunoglobulins; colostrum; and vitamin and mineral supplements, including but not limited to, zine chloride, magnesium chloride, pyridoxine, vitamin B-12, B complexes, folic acid, sodium ascorbate, and probiotics. Additional secondary therapies may include conventional chemotherapy, radiation therapy, and/or surgery.

In certain illustrative embodiments directed to treatment of glioblastoma (GBM), pancreatic cancer and other AMPAR-dependent types/forms of cancer, the PMP or other AMPAR antagonist is coordinately administered with temozolomide (TMZ). In related embodiments, the PMP or other AMPAR antagonist is coordinately administered with cisplatin to treat an AMPAR-dependent cancer, for example an AMPAR-positive pancreatic cancer. In other exemplary embodiments, an anti-cancer effective PMP or other AMPAR antagonist compound is coordinately administered with hydroxyurea to treat an AMPAR positive cancer. In other embodiments, the PMP or other AMPAR antagonist is coordinately administered with Carmustine (BCNU) to treat an AMPAR positive cancer.

In other coordinate methods and compositions, anti-cancer effective AMPAR antagonist administration is combined with a secondary anti-cancer agent or therapy, e.g., selected from a transcription inhibitor (e.g., Terameprocol), a telomere disrupting agent (e.g., TRF1 inhibitors such as ETP-4707), an inhibitor of a gene splicing protein (e.g., a PRMT5 inhibitor), an indoleamine 2, 3, dioxegenase (IDO) inhibitor, lapatinib ditosylate enzyme blocker, anti-cancer antibodies, antibody fragments and related “biologics”, for example Adavosertib, tumor treating fields, and radiation, alone or in any combination with other secondary or adjunctive cancer agents and treatments described herein.

In related embodiments, coordinate anti-cancer treatment methods of the invention can include coordinate administration of one or more anti-cancer AMPAR antagonist compounds, such as PMP, in combination with one or a plurality of any combination of secondary therapeutic agent(s) or therapy(ies) selected from: NMDA antagonists such as memantine for the treatment of various cancer; anti PD-1/PDL-1 therapy; CSF1R inhibitors such as PLX3397 and PLX5622; cannabinoid drugs; anti-malarials such as mefloquine, primaquine, chloroquine, hydroxychloroquine; Riluzole/troriluzole treatment; antihistamines such as clemastine; biguanides such as metformin or phenformin; anti-cancer biologics such as Pembrolizumab or Nivolumab; selective serotonin reuptake inhibitors (SSRIs); tricyclic antidepressants (TCAs); AMPA receptor positive allosteric modulators (Ampakines); levetiracetam (Keppra), and other agents, therapies and combinations contemplated herein.

Within exemplary embodiments, typical drug doses or combinatorial drug doses (median or average doses among a treated patient class) may include, for example, peramplanel administered at about 12 mg/day (exemplary range 5-50 mg/day), Memantine at about 20 mg/day (exemplary range 5-75 mg/day). Riluzole at about 50 mg/day (exemplary range 10-100 mg/day). PLX3397 at about 1000 mg/day (exemplary range 300 2500 mg/day), Anti-malarials at about 250 mg every other day (exemplary range 50-200 mg/day or every other day). Metformin at about 2 g/day (exemplary range 300 mg-4 g/day). Pembrolizumab at about 2 mg/kg every 3 weeks (exemplary range 0.05-10 mg/kg every 1-4 weeks), Nivolumab at about 3 mg/kg every 2 weeks (exemplary range 1-5 mg/kg every 1-3 weeks), Levetiracetam at about 500 mg twice a day. Clemastine fumarate at about 2.5 mg per day (exemplary range 1-5 mg per day), Escitalopram at about 20 mg/day (exemplary range 5-75 mg/day), sertraline at about 200 mg/day (exemplary range 50-800 mg/day), fluoxetine at about 20 mg/day (exemplary range 5-200 mg/day), Imipramine hydrochloride at about 25-50 mg/day (exemplary range 2-400 mg/day). Ampakines at about 900 mg/day, with high impact ampakines at about 100/day (exemplary range 25 mg-1.5 g/day); CBD at about 100-600 mg/day (exemplary range 20-1000 mg/day); THC at about 5-100 mg/day (exemplary range 1-800 mg/day); Ketamine/hydroxynorketamine at about 5-500 mg/day (exemplary range 1-1000 mg/day). Disulfiram at about 50-500 mg/day (exemplary range 20-1500 mg/day), or any combination of the foregoing drugs/doses.

In more detailed aspects of the invention employing coordinate treatment with Ampakines, operable ampakines can be selected from a wide variety of known ampakine compounds. Ampakines, while structurally diverse as a whole, show many shared structural and functional features within classes. Both between and within known ampakine classes, useful drug candidates operable within the anti-cancer methods and compositions of the invention can be identified, selected and proven effective according to the detailed teachings and guidance herein. Following these teachings, anti-cancer active ampakines can be selected among positive allosteric AMPA receptor modulators from within a variety of known ampakine groups. Among the ampakine classes from which operable ampakine candidates for use within the invention can be selected include ampakines generally classified as: sulfonamide compounds and derivatives, (bis)sulfonamide compounds and derivatives, N-substituted sulfonamide compounds and derivatives; heterocyclic sulfonamide compounds and derivatives; heterocyclyl sulfonamide compounds and derivatives; alkenyl sulfonamide compounds and derivatives; cycloalkenyl sulfonamide compounds and derivatives; cyclopentyl sulfonamide compounds and derivatives; cycloalkylfluoro sulfonamide compounds and; acetylenic sulfonamide compounds and derivatives; 2-propane-sulfonamide compounds and derivatives; 2-aminobenzenesulfonamide compounds and derivatives; benzoyl piperidine and benzoyl compounds and derivatives; pyrrolidine compounds and derivatives; benzoxazine ring compounds and derivatives; acylbenzoxazine compounds and derivatives; carbonylbenzoxazine compounds and derivatives; substituted 2,3-benzodiazepin-4-one compounds and derivatives; amidophosphate; monofluoralkyl compounds and derivatives; substituted quinazoline compounds and derivatives; quainoxaline compounds and derivatives; 2-ethoxy-4′-[3-(propane-2-sulfonylamino)-thiophen-2-yl]-biphenyl-4-carboxylic and derivatives; pyrrole and pyrazole compounds and derivatives; thiadiazine compounds and derivatives; benzofurazan compounds and derivatives; benzothiazide compounds and derivatives; substituted 5-oxo-5,6,7,8-tetrahydro-4H-1-benzopyran and benzothiopyran compounds and derivatives; benzoxazepine compounds and derivatives; among known classes of compounds comprising AMPA receptor modulator compounds prospectively useful within the invention.

According to the teachings and examples presented herein, anti-cancer effective ampakines effective within the invention are selected and characterized from among various structural classes of ampakines, for example, to demonstrate low impact convulsant risk and therapeutically effective anti-cancer activity. In illustrative embodiments provided herein, ampakines from the known class of benzofurazan ampakine compounds and derivatives (e.g., as disclosed in U.S. Pat. Nos. 6,110,935; and 6,313,115; and PCT Int'l Pub. No. WO9835950) were screened and developed to identify operable drug candidates within the compositions and methods of the invention. From these investigations exemplary anti-cancer benzofurazan candidates 1-(benzofurazan-5-ylcarbonyl)-4,4-difluoropiperidine, and 4-(benofurazan-5-ylcarbonyl), and 1-(benzofuran-5-ylcarbonyl)morpholine. Within additional compositions and methods of the invention, low impact ampakines are selected for combinatorial treatment methods of the invention from another ampakine group known collectively as “di-substituted amide ampakines.” These ampakines were first described by Cortex (now RespireRx), as detailed in U.S. Ser. No. 12/451,515, US Publication No. US2010/0120764, and PCT/US/2008/00627 (incorporated herein in their entirety, for all purposes). Exemplary di-substituted amide ampakines for use within the invention include N-Methyl-N-tetrahydro-2H-pyran-4-yl-[2,1,3]-benzoxadiazole-5-carboxamide (“CX1739”), Trans-4-[(2,1,3-benzoxadiazol-5-ylcarbonyl)(methyl)amino]cyclohexyl glycinate hydrochloride (CX 1942); and, N-(4-trans-hydroxycyclohexyl)-N-methyl-[2,1,3]-benzoxadiazole-5-carboxamide (CX1763). Within related embodiments of the invention, useful low impact, anti-cancer ampakines are selected and demonstrated to be active according to the teachings herein, having the exemplary ampakine structure I, below:

wherein:
W is oxygen, sulfur or CH═CH;
X, Y and Z are independently selected from the group consisting of —N, or —CR, wherein:
R is H, —Br, —Cl, —F, —CN, —NO₂, —OR¹, —SR¹, —NR¹₂, —C₁-C₆ branched or un-branched alkyl, which may be un-substituted or substituted,
wherein:
R¹ is H, —C₁-C₆ branched or un-branched alkyl which, may be un-substituted or substituted.
F═O or S.
A is H, or —C₁-C₆ branched or un-branched alkyl, which may be un-substituted or substituted, —C₂-C₆ branched or un-branched alkenyl, which may be un-substituted or substituted, —C₂-C₆ branched or un-branched alkynyl, which may be un-substituted or substituted, —C₃-C₇ cycloalkyl which may be un-substituted or substituted, —C₃-C₇ alkylcycloalkyl which may be un-substituted or substituted, aryl or heterocycle which may be un-substituted or substituted, alkylaryl which may be un-substituted or substituted, alkylheterocycle which may be un-substituted or substituted n=0, 1, 2.3, 4, 5, or 6;

is a —C₃-C₇ cycloalkyl, which may be un-substituted or substituted, a —C₄-C₇ azacycloalkyl, which may be un-substituted or substituted, a C₇-C₁₀ bicycloalkyl which may be un-substituted or substituted, a —C₇-C₁₀ azabicycloalkyl which may be un-substituted or substituted, aryl which may be un-substituted or substituted or a heterocycle which may be un-substituted or substituted;
B is —C═, C—R^{a}, O, N, S, C═O, S═O or SO₂,
R^{a} is H, a halogen (preferably F), OH, O-alkyl, cyano, or a —C₁-C₆ alkyl group which is un-substituted or substituted and which optionally, forms a C₃-C₇ cycloalkyl group with D; and
D is absent when B is O, S, S═O, C═O or SO₂, or if present, is bonded to B when B is —C═, —C—R^{a} or N, and is H, a halogen (preferably F), OR^{b}, a —C₁-C₆ branched or un-branched alkyl, which may be un-substituted or substituted and which optionally, forms a C₃-C₇ cycloalkyl group with R^{a}, a —C₂-C₆ branched or un-branched alkenyl, which may be un-substituted or substituted, a —C₂-C₆ branched or un-branched alkynyl, which may be un-substituted or substituted, a —C₃-C₇ cycloalkyl which may be un-substituted or substituted, an aryl which may be un-substituted or substituted, a heterocycle which may be un-substituted or substituted, a —C₂-C₇ carboxyalkyl which may be un-substituted or substituted, a carboxyaryl which may be un-substituted or substituted, a carboxylheteroaryl which may be un-substituted or substituted, a —C₁-C₇ sulfonylalkyl which may be un-substituted or substituted, a sulfonylaryl which may be un-substituted or substituted or a sulfonylheteroaryl which may be un-substituted or substituted, or when B is —C—R^{a}, R^{a} and D optionally form a ═N—R^{c} or a ═N—OR^{c} group with B, wherein R^{c} is H or an unsubstituted or substituted C₁-C₇ alkyl group, or when B is —C—R^{a}, R^{a} and D optionally form a ═N—R^{c} or a ═N—OR^{c} group with B, wherein R^{c} is H or an unsubstituted or substituted C₁-C₇ alkyl group; and R^{b} is H, a —C₁-C₇ alkyl group which may be branched or un-branched, un-substituted or substituted or a —C₂-C₇ acyl group which may be un-substituted or substituted.

Other exemplary ampakines useful within the combinatorial methods herein include include compounds according to formula II below:

wherein:
A is —C₁-C₆ branched or un-branched alkyl, which may be un-substituted or substituted, a C₃-C₇ cycloalkyl which may be un-substituted or substituted;
n is 0, 1, 2, or 3;
B is C—R^{a}, O or C═O;
R^{a} is H, F, —OH or alkyl and
D is absent (when B is O), is H or OH when R^{a} is H or alkyl, or is F when R^{a} is F, or a pharmaceutically acceptable salt, solvate, or polymorph thereof.

Yet additional exemplary ampakines for use within the invention include compounds according to formula Ill below:

wherein:
A is a C₁-C₆ alkyl which may be substituted or un-substituted;
B is C—R^{a}, O or C═O;
R^{a} is H, F, —OH or alkyl and
D is absent (when B is O), is H or OH when R^{a} is H or alkyl, or is F when R^{a} is F, or a pharmaceutically acceptable salt, solvate, or poly morph thereof.

Other exemplary ampakines for use within the invention include compounds according to formula IV below:

wherein:
A is a C₁-C₆ alkyl which may be substituted or un-substituted,
n is 0, 1 or 2, or a pharmaceutically acceptable salt, solvate, or polymorph thereof.

Other exemplary embodiments include compounds according to formula V below:

wherein:
A is a C₁-C₆ alkyl which may be substituted or un-substituted,
R¹ is H, F, or C₁-C₄ alkyl.
R is H, F, CN, a heterocycle which may be substituted or un-substituted or OR³,
R³ is H, C₁-C₆ alkyl which may be substituted or un-substituted, or a pharmaceutically acceptable salt, solvate, or polymorph thereof.

Other exemplary ampakines for use within the invention include compounds according to formula VI below:

wherein:
A is a C₁-C₆ alkyl which may be substituted or un-substituted,
R is H, or C₁-C₄ alkyl, or a pharmaceutically acceptable salt, solvate, or polymorph thereof.

Other exemplary ampakines for use within the invention include compounds according to formula VII below:

wherein:
B is C—R^{a}, O or C═O;
R^{a} is H, F, —OH or alkyl and
D is absent (when B is O), is H or OH when R^{a} is H or alkyl, or is F when R^{a} is F, or a pharmaceutically acceptable salt, solvate, or polymorph thereof.

Other exemplary ampakines for use within the invention include compounds according to formula VIII below:

wherein:
B is C—R^{a}, O or C═O;
R^{a} is H, F, —OH or alkyl and
D is absent (when B is O), is H or OH when R^{a} is H or alkyl, or is F when R^{a} is F, or a pharmaceutically acceptable salt, solvate, or polymorph thereof.

Other exemplary ampakines for use within the invention include compounds according to formula IX below:

wherein:
A is a C₁-C₆ alkyl which may be substituted or un-substituted, R¹ is H, or C₁-C₄ alkyl,
R² is H, or a C₁-C₆ alkyl which may be substituted or un-substituted,
R³ is H, or a C₁-C₆ alkyl which may be substituted or un-substituted,
R⁴ is H, or a C₁-C₆ alkyl which may be substituted or un-substituted, or a pharmaceutically acceptable salt, solvate, or polymorph thereof.

In more detailed embodiments, anti-cancer active compounds are selected from compounds of Formulas I-IX above that are already isolated and characterized, selected from: N-Cycloheptyl-N-methyl-[2,1,3]-benzoxadiazole-5-carboxamide; N-(4,4-Dimethylcyclohexyl-N-methyl-[2,1,3]-benzoxadiazole-5-carboxamide; N-Methyl-N-spiro[2.5]oct-6-yl-[2,1,3]-benzoxadiazole-5-carboxamide; N-Cyclohexyl-N-methyl-[2,1,3]-benzoxadiazole-5-carboxamide; N-Cyclopentyl-N-methyl-[2,1,3]-benzoxadiazole-5-carboxamide; N-Cyclobutyl-N-methyl-[2,1,3]-benzoxadiazole-5-carboxamide; N-Cyclohexyl-[2,1,3]-benzoxadiazole-5-carboxamide; N-Cyclopentyl-[2,1,3]-benzoxadiazole-5-carboxamide; N-Cyclobutyl-[2,1,3]-benzoxadiazole-5-carboxamide; N-(cis-4-Cyanocyclohexyl)-N-methyl-[2,1,3]-benzoxadiazole-5-carboxamide; N-(rans-4-Cyanocyclohexyl)-N-methyl-[2,1,3]-benzoxadiazole-5-carboxamide; N-Methyl-N-tetrahydro-2H-pyran-4-yl-[2,1,3]-benzoxadiazole-5-carboxamide (CX1739); N-D₃-Methyl-N-tetrahydro-2H-pyran-4-yl-[2,1,3]-benzoxadiazole-5-carboxamide; N-(Tetrahydro-2H-pyran-4-yl)-[2,1,3]-benzoxadiazole-5-carboxamide; N-(Tetrahydro-2H-pyran-3-yl)-[2,1,3]-benzoxadiazole-5-carboxamide; N-Methyl-N-(tetrahydro-2H-pyran-3-yl)-[2,1,3]-benzoxadiazole-5-carboxamide; N-Ethyl-N-tetrahydro-2H-pyran-4-yl-[2,1,3]-benzoxadiazole-5-carboxamide; N-Cyclohexyl-N-ethyl-[2,1,3]-benzoxadiazole-5-carboxamide; N-(Cyclohexylmethyl)-N-methyl-[2,1,3]-benzoxadiazole-5-carboxamide; N-Benzyl-N-methyl-[2,1,3]-benzoxadiazole-5-carboxamide; N-Methyl-N-(tetrahydrofuran-2-ylmethyl)-[2,1,3]-benzoxadiazole-5-carboxamide; N-Methyl-N-pyridin-3-yl-[2,1,3]-benzoxadiazole-5-carboxamide; N-Methyl-N-phenyl-[2,1,3]-benzoxadiazole-5-carboxamide N-Cyclopropyl-N-tetrahydro-2H-pyran-4-yl-[2,1,3]-benzoxadiazole-5-carboxamide N-Tetrahydro-2H-pyran-4-yl-N-(2,2,2-trifluoroethyl)-[2,1,3]-benzoxadiazole-5-carboxamide; tert-Butyl-4-[([2,1,3]-benzoxadiazol-5-ylcarbonyl)(methyl)amino]piperidine-1-carboxylate; N-Methyl-N-piperidin-4-yl-[2,1,3]-benzoxadiazole-5-carboxamide hydrochloride; N-Methyl-N-(1-methylpiperidin-4-yl)-[2,1,3]-benzoxadiazole-5-carboxamide; N-(1-Acetylpiperidin-4-yl)-N-methyl-[2,1,3]-benzoxadiazole-5-carboxamide; N-(1-Formylpiperidin-4-yl)-N-methyl-[2,1,3]-benzoxadiazole-5-carboxamide; N-Methyl-N-[1-(methylsulfonyl]piperidin-4-yl)-[2,1,3]-benzoxadiazole-5-carboxamide; N-Methyl-N-(tetrahydro-2H-pyran-4-yl)-[2,1,3]-benzothiadiazole-5-carboxamide; N-Methyl-N-(tetrahydro-2H-thiopyran-4-yl)-[2,1,3]-benzoxadiazole-5-carboxamide; N-Methyl-N-(1-oxidotetrahydro-2H-thiopyran-4-yl)-[2,1,3]-benzoxadiazole-5-carboxamide; N-Methyl-N-(1,1-dioxidotetrahydro-2H-thiopyran-4-yl)-[2,1,3]-benzoxadiazole-5-carboxamide; N-Methyl-N-tetrahydro-2H-pyran-4-ylquinoxaline-6-carboxamide; N-Methyl-N-(4-oxocyclohexyl)-[2,1,3]-benzoxadiazole-5-carboxamide; N-[4-(Hydroxyimino)cyclohexyl]-N-methyl-[2,1,3]-benzoxadiazole-5-carboxamide; N-[4-(Methoxyimino)cyclohexyl]-N-methyl-[2,1,3]-benzoxadiazole-5-carboxamide; N-(4,4-Difluorocyclohexyl)-N-methyl-[2,1,3]-benzoxadiazole-5-carboxamide; N-(4-fluorocyclohex-3-en-1-yl)-N-methyl-[2,1,3]-benzoxadiazole-5-carboxamide; N-(4-trans-Hydroxycyclohexyl)-[2,1,3]-benzoxadiazole-5-carboxamide; N-(trans-4-Hydroxy-4-methylcyclohexyl)-[2,1,3]-benzoxadiazole-5-carboxamide; N-(cis-4-Hydroxy-4-methylcyclohexyl)-N-methyl-[2,1,3]-benzoxadiazole-5-carboxamide; N-(trans-4-Hydroxy-4-methylcyclohexyl)-N-methyl-[2,1,3]-benzoxadiazole-5-carboxamide; N-(cis-4-Hydroxy-4-ethylcyclohexyl)-N-methyl-[2,1,3]-benzoxadiazole-5-carboxamide; N-(trans-4-Hydroxy-4-ethylcyclohexyl)-N-methyl-[2,1,3]-benzoxadiazole-5-carboxamide; N-(cis-4-Ethynyl-4-hydroxycyclohexyl)-N-methyl-[2,1,3]-benzoxadiazole-5-carboxamide; N-(cis-4-But-3-en-1-yl-4-hydroxycyclohexyl)-N-methyl-[2,1,3]-benzoxadiazole-5-carboxamide; N-(trans-4-But-3-en-1-yl-4-hydroxycyclohexyl)-N-methyl-[2,1,3]-benzoxadiazole-5-carboxamide; N-(4-trans-Hydroxycyclohexyl)-N-methyl-[2,1,3]-benzoxadiazole-5-carboxamide (CX1763); N-(4-trans-Hydroxycyclohexyl)-N-D₃-methyl-[2,1,3]-benzoxadiazole-5-carboxamide; N-(trans-4-Methoxycyclohexyl)-N-methyl-[2,1,3]-benzoxadiazole-5-carboxamide; N-(trans-4-Methoxycyclohexyl)-N-methyl-[2,1,3]-benzoxadiazole-5-carbothioamide; N-(4-cis-Hydroxycyclohexyl)-N-methyl-[2,1,3]-benzoxadiazole-5-carboxamide; N-Methyl-N-[trans-4-(2H-tetrazol-2-yl)cyclohexyl]-[2,1,3]-benzoxadiazole-5-carboxamide; N-(trans-4-Azidocyclohexyl)-N-methyl-[2,1,3]-benzoxadiazole-5-carboxamide; N-(trans-4-Aminocyclohexyl)-N-methyl-[2,1,3]-benzoxadiazole-5-carboxamide; N-(cis-3-Hydroxycyclohexyl)-N-methyl-[2,1,3]-benzoxadiazole-5-carboxamide; N-(trans-3-Hydroxycyclohexyl)-N-methyl-[2,1,3]-benzoxadiazole-5-carboxamide; N-Methyl-N-(3-oxocyclohexyl)-[2,1,3]-benzoxadiazole-5-carboxamide; N-Methyl-N-(3,3-difluorocyclohexyl)-[2,1,3]-benzoxadiazole-5-carboxamide; N-(2-Hydroxycyclohexyl)-N-methyl-[2,1,3]-benzoxadiazole-5-carboxamide; N-Methyl-N-(2-oxocyclohexyl)-[2,1,3]-benzoxadiazole-5-carboxamide; N-Methyl-N-(2,2-difluorocyclohexyl)-[2,1,3]-benzoxadiazole-5-carboxamide; N-(2-Hydroxytetrahydro-2H-pyran-4-yl)-[2,1,3]-benzoxadiazole-5-carboxamide; N-(2-oxotetrahydro-2H-pyran-4-yl)-[2,1,3]-benzoxadiazole-5-carboxamide; N-Methyl-N-(2-oxotetrahydro-2H-pyran-4-yl)-[2,1,3]-benzoxadiazole-5-carboxamide; N-(2-Hydroxytetrahydro-2H-pyran-4-yl)-N-methyl-[2,1,3]-benzoxadiazole-5-carboxamide; trans-4-[(2,1,3-Benzoxadiazol-5-ylcarbonyl)(methyl)amino]cyclohexyl N,N-dimethyl glycinate hydrochloride; trans-4-[(2,1,3-Benzoxadiazol-5-ylcarbonyl)(methyl)amino]cyclohexyl L-alaninate hydrochloride; N-(R)-Tetrahydrofuran-3-yl-[2,1,3]-benzoxadiazole-5-carboxamide; N-Methyl-N-(R)-tetrahydrofuran-3-yl-[2,1,3]-benzoxadiazole-5-carboxamide; trans-4-[(2,1,3-Benzoxadiazol-5-ylcarbonyl)(methyl)amino]cyclohexyl L-glycinate hydrochloride; N-2-(4-Morpholinyl)ethyl-[2,1,3]-benzoxadiazole-5-carboxamide; N-Methyl-N-2-(4-morpholinyl)ethyl-[2,1,3]-benzoxadiazole-5-carboxamide hydrochloride; N-Methyl-N-tetrahydro-2H-pyran-4-yl-[2,1,3]-benzoxadiazole-5-carbothioamide (CX 1739); trans-4-[(2,1,3-Benzoxadiazol-5-ylcarbonyl)(methyl)amino]cyclohexyl L-valinate hydrochloride; trans-4-[(2,1,3-Benzoxadiazol-5-ylcarbonyl)(methyl)amino]-1-methylcyclohexyl N,N-dimethyl glycinate hydrochloride; N-Methyl-N-tetrahydro-2H-pyran-4-ylmethyl-[2,1,3]-benzoxadiazole-5-carboxamide; and trans-4-[(2,1,3-Benzoxadiazol-5-ylcarbonyl)(methyl)amino]-1-methylcyclohexyl glycinate hydrochloride (CX1942).

Within additional compositions and methods of the invention, low impact ampakines are employed in the methods and compositions of the invention, selected from yet additional ampakine groups, including “bicyclic amide ampakines.” Among the many bicyclic amide ampakines candidates for use within the invention are the following exemplary species: 8-Azabicyclo[3,2,1]oct-8-yl([2,1,3]-benzoxadiazol-5-yl)methanone; 8-([2,1,3]-Benzoxadiazol-5-ylcarbonyl)-8-azabicyclo[3,2,1]octan-3-one; [2,1,3]-Benzoxadiazol-5-yl(3,3-difluoro-8-azabicyclo[3,2,1]oct-8-yl)methanone; endo-[2,1,3]-Benzoxadiazol-5-yl(3-hydroxy-8-azabicyclo[3,2,1]oct-8-yl)methanone; exo-[2,1,3]-Benzoxadiazol-5-yl(3-hydroxy-8-azabicyclo[3,2,1]oct-8-yl)methanone; 2-Azabicyclo[2,2,1]hept-2-yl([2,1,3-benzoxadiazol-5-yl)methanone; 1-Azabicyclo[2.2.1]hept-1-yl(2,1,3]-benzoxadiazol-5-yl)methanone; 2-Azabicyclo[2,2,2]oct-2-yl([2,1,3]-benzoxadiazol-5-yl)methanone; [2,1,3]-Benzoxadiazol-5-yl(5,6-dichloro-2-azabicyclo[2,2,1]hept-2-yl)methanone. Additional bicyclic amide ampakines for prospective use within the anti-cancer methods and compositions of the invention include, but are not limited to, the following exemplary species; [2,1,3]-Benzoxadiazol-5-yl(3-fluoro-8-azabicyclo[3,2,1]oct-2-en-8-yl)methanone; 2-Azabicyclo[2,2,1]hept-5-en-2-yl([2,1,3]-benzoxadiazol-5-yl)methanone; R-2-Azabicyclo[2,2,1]hept-5-en-2-yl([2,1,3]-benzoxadiazol-5-yl)methanone; S-2-Azabicyclo[2,2,1]hept-5-en-2-yl([2,1,3]-benzoxadiazol-5-yl)methanone; and [2,1,3]-Benzoxadiazol-5-yl(2-oxa-5azabicyclo[2,2,1]hept-5-yl)methanone.

Yet additional ampakine compounds flor use within the invention will be selected according to the teachings herein, using known AMPA receptor modulator compounds, reagents, preparative methods and other tools as disclosed in the following publications, each of which is incorporated herein for all purposes; PCT Int'l Pub. No. WO 94/02475 and related U.S. Pat. Nos. 5,773,434, 5,488,049, 5,650,409, 5,736,543, 5,747,492, 5,773,434, 5,891,876, 6,030,968, 6,274,600, 6,329,368, 6,943,159, and 7,026,475; U.S. Pat. Pub. No. 20020055508; U.S. Pat. Nos. 6,174,922, 6,303,816, 6,358,981, 6,362,230, 6,500,865, 6,515,026, and 6,552,086; PCT Int'l Pub. Nos. WO 0190057, WO 0190056, WO 0168592, WO 0196289, WO 02098846, WO 0006157, WO 9833496, WO 0006083, WO 0006148, WO 0006149, WO 9943285, and WO 9833496; WO 0194306; U.S. Pat. No. 6,525,099 and PCT Int'l Pub. No. WO 0006537; U.S. Pat. No. 6,355,655 and PCT Int'l Pub. Nos. WO0214294, WO0214275, and WO0006159; U.S. Pat. No. 6,358,982 and PCT Int'l Pub. No. WO0006158; U.S. Pat. No. 6,387,954 and PCT Int'l Pub. No. WO0006539; PCT Int'l Pub. No. WO02098847; U.S. Pat. No. 6,639,107 and PCT Int'l Pub. No. WO0142203; PCI Int'l Pub. No. WO0232858; PCT Int'l Pub. No. WO0218329; U.S. Pat. No. 6,596,716 and PCI Int'l Pub. Nos; WO2006087169, WO2006015827, WO2006015828, WO2006015829, WO2007090840, and WO2007090841; WO02089734; U.S. Pat. Nos. 5,650,409, 5,747,492, 5,783,587, 5,852,008, and 6,274,600; U.S. Pat. Nos. 5,736,543, 5,962,447, 5,985,871, and PCT Int'l Pub. Nos. WO 9736907 and WO9933469; U.S. Pat. No. 6,124,278, and PCT Int'l Pub. No. WO 9951240; PCT Int'l Pub. No. WO03045315; U.S. Pat. Nos. 5,891,871; 6,110,935 and 6,313,115, and PCT Int'l Pub. No. WO9835950; PCT Int'l Pub. No. WO 9812185; PCT Int'l Pub. No. WO0075123; U.S. Pat. No. 6,521,605 and PCT Int'l Pub. No. WO0006176; PCT Int'l Pub. No. WO 0066546; PCT Int'l Pub. No. WO 9944612; PCT Int'l Pub. No. WO2007060144; U.S. Pat. Pub. No. 20060276532; U.S. Pat. Pub. No. 20070066573; U.S. Pat. Pub. No. 20070004709; U.S. Pat. Pub. No. 20040171605; PCT Int'l Pub. Nos. WO 9942456, WO 0006156, and WO 0157045, and U.S. Pat. No. 6,617,351.

Additional description and background pertaining also to specific positive allosteric AMPA receptor modulators, their preparation, use and selection within the compositions and methods of the invention, is provided in the following references, incorporated herein in toto for all purposes; For benzofurazan compounds-PCT patent application PCT/US98/02713, U.S. patent application Ser. No. 08/800,108, now U.S. Pat. No. 6,110,935, U.S. patent application Ser. No. 09/355,139, now U.S. Pat. No. 6,313,115, U.S. patent application Ser. No. 09/834,349; U.S. patent application Ser. No. 09/845,128, now U.S. Pat. No. 6,730,677; For di-substituted amide ampakines-PCT patent application PCT/US2008/006271, U.S. patent application Ser. No. 12/451,515, now U.S. Pat. No. 8,013,003, U.S. patent application Ser. No. 13/226,146, now issued U.S. Pat. No. 8,404,682, and U.S. patent application Ser. No. 13/755,210, now issued U.S. Pat. No. 8,642,633; For bicyclic amide ampakines-PCT patent application PCT/US2008/009508, United States Provisional patent application, Ser. No. 60/964,362; U.S. patent application Ser. No. 12/657,908, now U.S. Pat. No. 8,119,632, U.S. patent application Ser. No. 12/733,073, now U.S. Pat. No. 8,263,591, U.S. patent application Ser. No. 13/348,171, now U.S. Pat. No. 8,507,482, U.S. patent application Ser. No. 13/557,681, U.S. patent application Ser. No. 12/657,924, now U.S. Pat. No. 8,168,632, PCT patent application PCT/US2010/000255, and U.S. Provisional patent application, Ser. No. 61/206,642; For bicyclic amide ampakines-PCT patent application PCT/US2010/000254, and U.S. Provisional patent application, Ser. No. 61/206,642; For 3-Substituted-[1,2,3]Benzotriazinone ampakines-PCT Patent application PCT/US2007/026415, United States Provisional patent application, Ser. No. 60/878,626. United States patent application. Ser. No. 12/448,770, PCT patent application PCT/US2007/026416, United States Provisional application, Ser. No. 60/878,503, U.S. Provisional patent application, Ser. No. 60/921,433, and U.S. patent application Ser. No. 12/448,784, now U.S. Pat. No. 8,173,644; For 3-substituted 1,2,3-triazin-4-one and 3-substituted 1,3-pyrimidinone ampakines-PCT patent application PCT/US2008/010877. United States Provisional patent application. Ser. No. 60/994,548, and U.S. patent application Ser. No. 12/733,822; For benzoxazine ampakines-PCT patent application PCI/US98/27027, and U.S. patent application Ser. No. 08/998,300, now U.S. Pat. No. 5,985,871; for Acylbenzoxazines ampakines-PCT patent application, Serial No. PCT/US99/07325, and U.S. patent application Ser. No. 09/054,916, now U.S. Pat. No. 6,124,278; for Benzoyl Piperidine/Pyrrolidine ampakines PCT patent application PCT/US96/07607, and U.S. patent application Ser. No. 08/458,967, filed 2 Jun. 1995, now U.S. Pat. No. 5,650,409; For benzoxazine ampakines-PCT patent application, Serial No. PCT/US97/05184. U.S. patent application Ser. No. 08/624,335, now U.S. Pat. No. 5,736,543, PCT patent application PCT/US93/06916, and U.S. patent application Ser. No. 07/919,512, now U.S. Pat. No. 5,962,447; and for carbonylbenzoxazine ampakines-PCT patent application PCT/US02/37646, United States Provisional patent application, Ser. No. 60/333,334, and U.S. patent application Ser. No. 10/495,049, now U.S. Pat. No. 7,799,913. Each of the foregoing classes and distinct structural groups of ampakine compounds disclosed in the above references are suitable for evaluation to determine operability within the methods and compositions of the invention. Persons of ordinary skill in the art will recognize that these various compound groups, while being structurally diverse, share common functional characteristics of positive allosteric AMPA receptor modulation, as described here, and that because of these common functional characteristics, the compounds can be evaluated and determined for their operability according to the inventive discoveries and teachings herein. According to the Examples and other guidance provided here, anti-cancer effective ampakines, for example, can be selected and demonstrated for beneficial, clinical use without undue experimentation.

To practice coordinate administration methods of the invention, the anti-cancer effective AMPAR antagonist compound is co-administered, simultaneously or sequentially, in a coordinate treatment protocol with one or more of the secondary or adjunctive therapeutic agents contemplated herein. Thus, in certain embodiments the anti-cancer effective AMPAR antagonist compound is administered coordinately with a conventional cancer chemotherapeutic agent using separate formulations or a combinatorial formulation. Coordinate administration may be done simultaneously or sequentially in either order, and there may be a time period while only one or both (or all) active therapeutic agents individually and/or collectively exert their therapeutic activities. A distinguishing aspect of all such coordinate treatment methods is that the anti-cancer effective AMPAR antagonist compound exerts at least some measurably distinct anti-cancer therapeutic activity, yielding a distinct clinical response, in addition to any complementary clinical response provided by the secondary or adjunctive therapeutic agent. Often, the coordinate administration of the anti-cancer effective AMPAR antagonist compound with the secondary or adjunctive therapeutic agent will yield improved anti-cancer therapeutic or prophylactic results in the subject beyond a therapeutic or prophylactic effect elicited by the secondary or adjunctive therapeutic agent alone, which benefit contemplates both direct effects, as well as indirect effects.

The anti-cancer effective AMPAR antagonist compounds and pharmaceutical compositions of the present invention may be administered by any means that achieve the contemplated anti-cancer therapeutic or prophylactic purpose. Suitable routes of administration for the compositions of the invention include, but are not limited to, oral, buccal, nasal, aerosol, topical, transdermal, mucosal, injectable, and intravenous, as well as all other practicable delivery routes, devices and methods.

The anti-cancer effective AMPAR antagonist compounds of the present invention may be formulated with a pharmaceutically acceptable carrier appropriate for the particular mode of administration employed. Dosage forms of the compositions of the invention include excipients recognized in the art of pharmaceutical compounding as being suitable for the preparation of dosage units as discussed herein. Such excipients include, without limitation, solvates, buffers, binders, fillers, lubricants, emulsifiers, suspending agents, sweeteners, flavorings, preservatives, wetting agents, disintegrants, effervescent agents and other conventional pharmaceutical excipients and additives.

Anti-cancer effective AMPAR antagonist compounds of the invention will often be formulated and administered in an oral dosage form, optionally in combination with a carrier and/or other additive(s). Suitable carriers for pharmaceutical formulation of oral dosage forms include, for example, microcrystalline cellulose, lactose, sucrose, fructose, glucose, dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms include ingestible and sublingual liquids, tablets, capsules, and films, among other options, which may be prepared by any conventional method known in the art, optionally including additional ingredients such as release modifying agents, glidants, compression aides, disintegrants, lubricants, binders, flavor enhancers, sweeteners and/or preservatives (e.g., stearic acid, magnesium stearate, talc, calcium stearate, hydrogenated vegetable oils, sodium benzoate, leucine carbowax, magnesium lauryl sulfate, colloidal silicon dioxide, glyceryl monostearate, colloidal silica, silicon dioxide, and glyceryl monostearate). Oral dosage forms may further include an enteric coating that dissolves after passing through the stomach, for example, a polymer agent, methacrylate copolymer, cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methyl vinyl ether and maleic anhydride (Gantrez ES series), and natural resins such as zein, shellac and copal collophorium.

If desired, oral, mucosal, gastric, transdermal, topical and injectable compositions of the invention can be administered in a controlled release form by use of such well known technologies as slow release carriers and controlled release agents.

In certain embodiments the anti-cancer effective AMPAR antagonist compound is administered to patients in an injectable or intravenous (iv) formulation and delivery mode. In illustrative aspects a therapeutic unit dosage of PMP is formulated in a physiological solution amenable for injection or iv delivery to human subjects, for example in an aqueous buffered solution such as saline. Alternative formulations of anti-cancer effective AMPAR antagonist compounds for administration to patients intravenously, intramuscularly, subcutaneously or intraperitoneally can include nonaqueous sterile injectable solutions and optionally contain anti-oxidants, buffers, bacteriostats and/or solutes which render the formulation isotonic with the blood of the subject, as well as aqueous and non-aqueous sterile suspensions which may include suspending agents and/or thickening agents. Additional injectable compositions and formulations of the invention may include polymers and other controlled delivery additives or carriers for extended release following administration. Parenteral preparations may be solutions, dispersions or emulsions suitable for such administration. Extemporaneous injection solutions, emulsions and suspensions may be prepared from sterile powders, granules and tablets. Preferred unit dosage formulations are those containing a daily dose or unit, daily sub-dose, or an appropriate fraction thereof, of the anti-cancer effective AMPAR antagonist compound and/or active ingredient(s). In some embodiments, localized delivery of anti-cancer effective AMPAR antagonist compounds may be achieved by injecting the parenteral formulation directly into an area surrounding a cellular malignancy, directly into a tumor, into the vasculature supplying a malignancy itself, or into a pleural or peritoneal cavity or cerebrospinal compartment proximal or fluidly connected to a targeted malignancy.

In certain embodiments the methods and compositions of the invention may employ a pharmaceutically acceptable salt of an anti-cancer effective AMPAR antagonist compound, for example an acid addition or base salt of a PMP compound, derivative or analog. Examples of pharmaceutically acceptable addition salts include inorganic and organic acid addition salts. Suitable acid addition salts are formed from acids which form non-toxic salts, for example, hydrochloride, hydrobromide, hydroiodide, sulphate, hydrogen sulphate, nitrate, phosphate, and hydrogen phosphate salts. Additional pharmaceutically acceptable salts include, but are not limited to, metal salts such as sodium salts, potassium salts, cesium salts and the like; alkaline earth metals such as calcium salts, magnesium salts and the like; organic amine salts such as triethylamine salts, pyridine salts, picoline salts, ethanolamine salts, triethanolamine salts, dicyclohexylamine salts, N,N′-dibenzylethylenediamine salts and the like; organic acid salts such as acetate, citrate, lactate, succinate, tartrate, maleate, fumarate, mandelate, acetate, dichloroacetate, trifluoroacetate, oxalate, and formate salts; sulfonates such as methanesulfonate, benzenesulfonate, and p-toluenesulfonate salts; and amino acid salts such as arginate, asparginate, glutamate, tartrate, and gluconate salts. Suitable base salts are formed from bases that form non-toxic salts, for example aluminum, calcium, lithium, magnesium, potassium, sodium, zinc and diethanolamine salts. In related embodiments, optional salt forms of an anti-cancer effective AMPAR antagonist compound will yield enhanced properties, e.g., improved stability, solubility, tolerability, etc.

In other detailed embodiments, the methods and compositions of the invention employ prodrugs of the anti-cancer effective AMPAR antagonist compound, e.g., prodrugs of a PMP compound or derivative, or of an intermediary compound, or precursor compound of a PMP compound or derivative. As contemplated herein, prodrugs of anti-cancer effective AMPAR antagonist compounds can include the active compound reversibly linked (e.g., covalently bonded) to any carrier compound or moiety that functions to release the active anti-cancer effective AMPAR antagonist compound in vivo (for example to effectively mediate delivery of more active drug, to enhance in vivo half-life of the drug, or otherwise enhance pharmacokinetics or pharmacodynamics of the drug following administration. Examples of prodrugs useful within the invention include esters or amides with hydroxyalkyl or aminoalkyl as a substituent, among many other prodrug constructs known in the art.

The invention will also be understood to encompass methods and compositions comprising biologically active metabolites and in vivo conversion products of the anti-cancer effective AMPAR antagonist compound (either generated in vivo after administration of the compound, or directly administered in the form of the metabolite or conversion product itself). Such secondary active products may result for example from oxidation, reduction, hydrolysis, amidation, esterification and the like, of the administered compound, primarily due to enzymatic processes.

### Example I

### Dose-Dependent Anti-Cancer Activity of Exemplary AMPAR Antagonist Perampanel (PMP)

T9G (Glioblastoma) and Pane-1 (pancreatic adenocarcinoma) were obtained from ATCC. They were maintained in DMEM media (ATCC) and supplemented with 10% FBS (ATCC) and 1% Penicillin/Streptomycin and maintained in an incubator at 37° C. with 95% air and 5% CO₂.

Reagents

PMP was purchased from Medkoo and dissolved in DMSO. Temozolomide, cisplatin and glutamate were purchased from Sigma and dissolved in complete media on the day of treatment.

Cancer Cell Viability Assays T98G or Panc1 cells were seeded in quadruplicate at a density of 6,000 cells/well in complete DMEM and incubated overnight. T98G cells were then treated with increasing concentrations of PMP and temozolomide for 72 hours. Alternatively, panc1 cells were treated with PMP, cisplatin or glutamate for 48 hours. Following this incubation, 15 uL MTS solution (Promega) was added to each well and incubated for a further 2 hours. Plates were read at 490 nM using the ELx808 microplate reader. Absorbance values of wells with only media were subtracted out as background control. Data were normalized to vehicle-treated cells.

Data Analysis

Data were analyzed using Microsoft excel using a student's t-test. One-way ANOVA were also performed using Statplus. King's Synergy formula was used to look for synergistic interactions among PMP and chemotherapies. Alpha value was set at p=0.05.

### Results

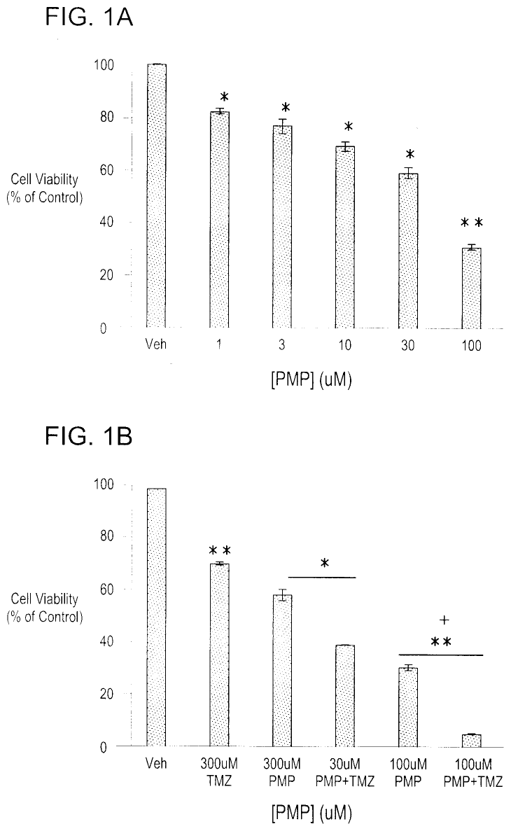

The data presented here unexpectedly reveal that AMPAR antagonists, exemplified by peramplanel (PMP), induce dose-dependent reductions in cell viability of T98G cells (FIG. 1a, ANOVA p<0.0001). The surprisingly potent oncolytic effects of peramplanel are mediated in part by antagonistic activity against AMPAR physiology in AMPAR positive cancer cell targets. Perampanel exhibited significant inhibitory activities against GBM cancer cell viability, even at concentrations of 1-10 uM, demonstrating the clinical utility of this drug at relevant plasma levels for effective cancer chemotherapy. PMP additively complements anti-cancer effects of temozolomide (TMZ) at 30 uM, and synergistically potentiates TMZ anti-cancer efficacy at 100 uM (FIG. 1b).

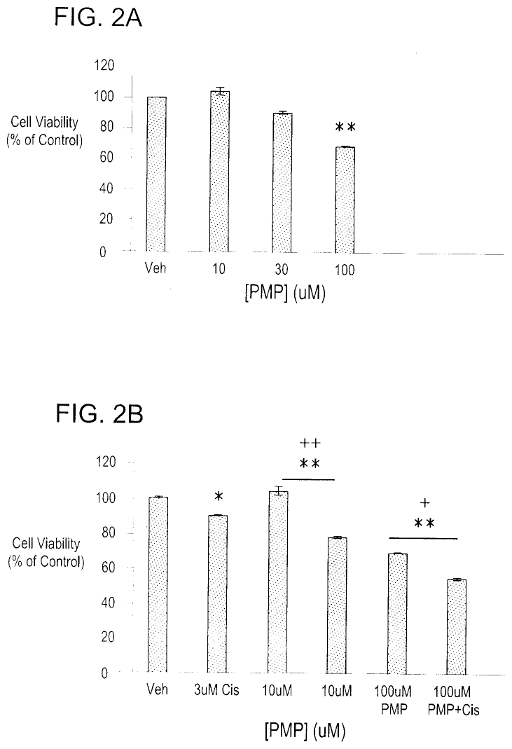

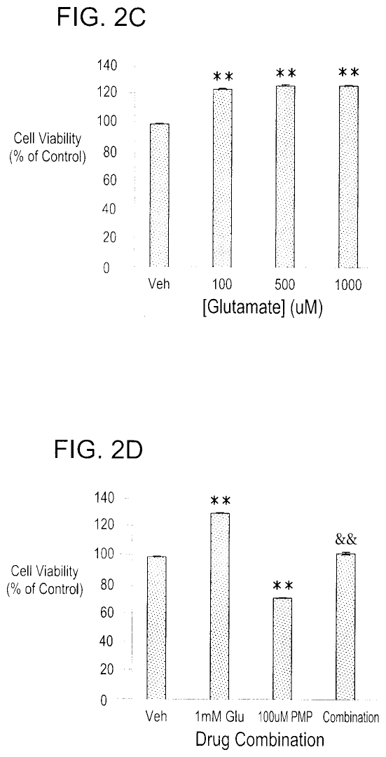

Previous work with pancreatic cancer has suggested that ampakines may be able to reduce cell viability. Thus, the results provided here demonstrating that PMP dose-dependently reduces Panc1 cell viability (ANOVA p=0.0013), with significant reduction of cell viability seen at 100 uM (FIG. 2a), are particularly surprising. Although 10 uM PMP did not appear to exert potent oncolytic effect alone at this concentration, it did significantly enhance oncolysis in combination with the drug cisplatin. Accordingly. PMP will be clinically effective to improve cancer treatments of this and other chemotherapeutic agents (FIG. 2b). 100 uM PMP and 3 uM cisplatin also synergistically reduced pancreatic cancer cell viability (FIG. 2b).

In yet additional working examples provided here, PMP effectively disrupted the oncogenic activity of exogenous glutamate, thereby inhibiting glutamate-potentiated pancreatic cancer activation (e.g., as demonstrated by impairment of cancer cell proliferation). In these studies, glutamate concentrations of 100-1000 uM elicited a dose-dependent acceleration of pancreatic cancer cell proliferation (FIG. 2c). In test samples panc1 cells were exposed to 1 mM glutamate for 15 minutes prior to the addition of PMP to cell culture media. Perampanel addition 15 minutes following glutamate exposure was sufficient to profoundly disrupt oncogenic activities of glutamate (FIG. 2d).

### Example II

### Dose-Dependent Anti-Cancer Activity of an Exemplary AMPAR Antagonist, Perampanel (PMP)

Potent anti-cancer efficacy of PMP compounds and other effective AMPAR antagonists is readily demonstrated using a range of animal models that are well known and widely accepted in the art as predictive of anti-cancer activity in humans. One such model employs subcutaneous xenografts of tumor cells into useful study animals such as mice, to study efficacy of candidate anti-cancer drugs in reducing growth or proliferation of xenografted tumor cells in test versus control subjects. These studies can include monitoring of a range of indicia of therapeutic efficiency, for example to demonstrate a dose-dependent decrease (e.g., based on average values observed in test versus control subjects) in xenografted tumor number, tumor size, tumor metastases, tissue histological and/or biochemical cancer markers (e.g., from biopsy or necropsy) blood cancer markers, mortality, etc. As used herein, cancer “markers” refers to any biomolecule, such as a growth factor, genetic regulatory protein, cytokine, hormone, receptor, etc., whose presence, expression, structure, level or activity is correlated with cancer incidence, severity, progression, or another etiologic or therapeutic factor indicative of cancer growth, metabolic activity, metastasis, etc.

In useful study protocols relating to central nervous system (CNS) cancers such as glioblastoma (GBM), conventional xenograft study designs may be modified to include intracranial xenografting, to better capitulate clinical conditions of GBM (see, for example, Ozawa et al., 2010). In one exemplary study protocol employed herein, modified from Ozawa et al., we employ T98G cells, a GBM cell line expressing the enzyme MGMT, which functions to repair DNA damage from temozolomide (TMZ), rendering this cell type intrinsically resistant to TMZ chemotherapy. These cells are engineered to express the bioluminescent enzyme luciferase to allow in vivo xenograft detection and quantification. A study total of 24 mice are used, divided into four study groups of 6 members per group. The mice are anesthetized using ketamine/zylazine on a warming plate to maintain core body temperature. Once anesthetized, the scalp is swabbed with chlorohexidine and a sagittal incision is made over the parieto-occipital bone, about 1 cm long on the left side. The exposed skull is cleaned using a cotton swab with 3% hydrogen peroxide. Xenograft cells are provided at a concentration of 300,000-500,000 cells in 3 uL serum-free media, and this cell suspension is drawn into a syringe and injected at a depth of 3 mm into the cortical tissue. The injection is carried out slowly, over a period of one minute, to localize the xenografted cells focally to specific brain region and prevent dissemination of the cells into the ventricles and spinal cord. After injection, the skull is cleaned with 3% hydrogen peroxide and sterile bone wax is to the incised skull defect. The scalp is drawn over the skull and stapled closed. Buprenorphine is optionally administered for post-operative pain relief, and recovery time is about 30 minutes.

One week after injection the study subjects are divided into 4 groups and bioluminescent monitoring of the xenografts begins. Group 1 mice receive placebo saline for the duration of the experiment. (Group 2 receives 20 mg/kg/day TMZ. Group 3 receives 5 or 10 mg/kg/day PMP depending on what dose produces a partial effect in monotherapy experiments. Group 4 is a combination group that receives both the TMZ and PMP treatments. Mice are bioluminescent monitored every 4 days during the study, for example using D-luciferin and an in vivo imaging system such as IVIS-200 (PerkinElmer, Inc., Norwalk Conn.) to measure bioluminescent photon release as a quantitative indicator of tumor growth.

These and related studies will demonstrate that PMP and other anti-cancer effective AMPAR antagonists according to the teachings herein potently prevent and treat AMPAR positive CNS cancers, including GBM, in mammalian subjects. Particular results will demonstrate a dose-dependent reduction in overall luminescence over an effective course of AMPAR antagonist treatment, correlated with reduced tumor size, reduced tumor cell number and/or reduced xenograft proliferative and/or metastatic capacity mediated by the anti-cancer AMPAR antagonist, for example PMP. PMP and other selected AMPAR antagonist will also significantly decrease tumor cell survival, viability and proliferation, and increase correlated indicia including time to tumor doubling and tripling, as well as subject survival (e.g., by time and/or numbers of subjects), in addition to mediating significant therapeutic benefits corresponding to all other anti-cancer activity indicators described herein above

In more detailed in vivo protocols. PMP will exhibit significant inhibitory activity against GBM xenograft cell viability, proliferative capacity, tumor growth and metastases at concentrations of 1-10 uM or greater, i.e., at plasma levels that are safe and effective for cancer chemotherapy.

In other detailed aspects, PMP will be shown to be combinatorially effective to complement anti-GBM effects of secondary anti-cancer drugs and treatments, for example temozolomide (TMZ). In certain embodiments, PMP will complement, potentiate or even synergistically enhance anti-cancer activities of other drugs, for example to significantly increase overall anti-cancer effects in combination with TMZ, compared to anti-cancer effects mediated by TMZ alone. In these embodiments the combinatorial use of PMP and TMZ, e.g., at therapeutic dosage levels of PMP between about 30 uM-100 uM, provides for enhanced efficacy of TMZ and lower TMZ dosages with reduced TMZ-associated side effects, an exemplary model of coordinate treatment that will be demonstrable across a range of combinations of AMPAR antagonists and secondary/adjunctive anti-cancer agents and therapies.

In related illustrative protocols the efficacy of anti-cancer AMPAR antagonists such as PMP is demonstrated in combinatorial usage with a PRMT5 inhibitor, such as EPZ015666. As recently reported by Braun et al (2017), high grade gliomas may be dependent on deletion of detained introns of oncogenic transcripts for sustained growth and survival. PRMT5 ensures proper splicing of these introns to become mature transcripts useful for production of various oncogenic proteins. Inhibition of PRMT5 with EP7015666 reportedly mediates oncostatic effects against GBM. In one illustrative study here, the foregoing intracranial xenograft study design is adapted to include one individual test group of 6 mice receiving 100 mg kg/da EPZ015666, one group treated with 10 mg/kg/day PMP, and a combinatorial group treated with both EPZ015666 and PMP. Bioluminescent imaging and other measures of anti-cancer efficacy will demonstrate that PMP is anti-cancer effective alone, and combinatorially effective (e.g., complementary, additive, potentiating or synergistic) in coordinate administration with EPZ015666.

In other illustrative protocols of the invention the efficacy of anti-cancer AMPAR antagonists such as PMP is demonstrated in combinatorial methods with tumor treating fields. Recent studies report that electrical fields using insulated electrodes applying frequencies of 200 kHz can inhibit cell cycle progression in GBM cells (see, e.g., Kirson et al, 2007; and Stupp et al. 2015). In an exemplary study here, the intracranial xenograft protocol is adapted to include one individual test group of 6 mice receiving an external insulated electrode closest to the area of the xenograft, applying a 200 kHz current for the duration of the study, one group treated with 10 mg/kg/day PMP, and a combinatorial group treated with both therapies. Bioluminescent imaging and other measures of anti-cancer efficacy will demonstrate that PMP is anti-cancer effective alone and combinatorially effective in coordinate administration with tumor treating fields.

In other exemplary protocols of the invention the efficacy of anti-cancer AMPAR antagonists such as PMP is demonstrated in combinatorial therapies employing proteins that interfere with telomere function of tumors, for example the TRF1 inhibitor ETP-47037. Due to rapid proliferation of most tumors, tumor cells are particularly vulnerable to DNA damage that can result in cell death. Telomeres are the caps of chromosomes made of repetitive DNA, which serve to prevent protein-coding DNA loss or damage during cell division. Several proteins are implicated in maintaining telomeres, one of which is a protein designated TRF1. Recent studies report that pharmacological or genetic ablation of this TRF1 reduces tumor formation and growth in animal models (see, e.g., Bejarano et al. 2017). In one report, 75 mg/kg of ETP-47037 prevented tumor growth in mice. In an illustrative protocol here, the intracranial xenograft protocol above is adapted to include one test group of mice receiving a therapeutic dosage of 75 mg/kg of ETP-47037, one group treated with 10 mg/kg/day PMP, and a combinatorial group treated with both therapies. Bioluminescent imaging and other measures of anti-cancer efficacy will demonstrate that PMP is anti-cancer effective alone and combinatorially effective in coordinate administration with ETP-47037. Additional studies are contemplated to show that combinatorial treatment with PMP provides for lower dosing of the ETP-47037 to achieve the same or greater clinical benefits, with fewer side effects (e.g., wherein a comparable, partial anti-cancer effect as exhibited by 75 mg/kg is observed in combination with PMP at reduced effective dosages of ETP-47037 of 25-50 mg/kg or lower).

In another exemplary combination treatment model of the invention, the efficacy of anti-cancer AMPAR antagonists such as PMP is demonstrated in coordinate protocols with a transcription inhibitor, such as terameprocol. Terameprocol is a global transcription inhibitor that affects proliferation, apoptosis and drug resistance, currently being clinically evaluated for treatment of GBM (Grossman et al. 2012). In one representative study the intracranial xenograft study includes one test group of mice treated with 20 mg/kg/day Terameprocol, one group treated with 10 mg/kg/day PMP, and a combinatorial group treated with both therapies. Bioluminescent imaging and other measures of anti-cancer efficacy will demonstrate that PMP is anti-cancer effective alone and combinatorially effective in coordinate administration with Terameprocol. Additional studies will show that combinatorial treatment with PMP provides for lower dosing of Terameprocol to achieve the same or greater clinical benefits, with fewer side effects.

In yet additional combinatorial treatment methods of the invention, the efficacy of anti-cancer AMPAR antagonists such as PMP is demonstrated in coordinate protocols with NEK2 inhibitors. Recent studies report that EZH2 is vital for maintaining glioma stem cells, a subset of glioma cells that are responsible for chemo- and radiotherapy resistance due to their ability to regenerate new tumor cells after existing tumor cells are destroyed. NEK2 is responsible for guarding EZH2 against premature breakdown, allowing EZH2 to exert a longer and more robust oncogenic effect. Recent studies report that an inhibitor of NEK2, Cmp3a, exerts anti-cancer effects as demonstrated by prolongation of cancer survival time in mice (Wang et al. 2017). According to the modified study protocol here one group of mice receives 10 mg/kg/day Cmp3a, one group received 10 mg/kg/day PMP, and a combinatorial group is treated with both therapies. Bioluminescent imaging and other measures of anti-cancer efficacy will demonstrate that PMP is anti-cancer effective alone and combinatorially effective in coordinate administration with NEK2 inhibitors such as Cmp3a. Additional studies will show that combinatorial treatment with PMP provides for lower dosing of NEK3 inhibitors to achieve the same or greater clinical benefits, with fewer side effects.

### Example III

### Anti-Cancer Activity of AMPAR Antagonists in Combination with Standard of Care (SOC) Glioma Treatment

In exemplary clinical protocols of the invention, anti-cancer effective AMPAR antagonist treatment will be combined with secondary anti-cancer therapy comprising standard of care (SOC) glioma treatments. In one illustrative example, patients are initially treated with SOC maximal safe surgical resection, followed by an aggressive SOC chemoradiation protocol. For the first 6 weeks of treatment, patients receive 75 mg/m2/day of Temozolomide (Temodar) starting one hour prior to radiation treatment. Patients additionally receive 200 cGy focal radiation per day for the first five days of the week over a 6 week timespan (30 fractions), for a total of 60 Gy radiation. Radiation targets the tumor area as well as surrounding edema plus a 1 cm margin, 1 month after the last radiation treatment, patients are administered 150-200 mg/m2/day temozolomide for the first 5 days of every month followed by 3 weeks rest as well as antiemetic prophylaxis treatment as needed. Treatments are stopped if platelet count drops below 100.000/uL, or if there is evidence of disease progression or severe treatment-related toxicity (Grossman et al. 2009). In combination with the S(X glioma treatment outlined above, patients receive 8 mg peramplanel orally 1 hour prior to the first radiation session. Perampanel is further administered once per day, and weekly titrated up 2 mg/day until patients receive the maximally tolerated approved dose (MTD) of 12 mg (Gidal et al. 2015) (though this range can be adjusted up or down based on patient-specific tolerance and other clinical factors determined by the managing physician). Within illustrative methods for treating GBM, patients receive the MTD throughout an initial 6-week treatment period, during the 1 month SOC rest period, and while patients are taking maintenance Temodar. Patients are maintained on perampanel treatment as determined by the managing physician, unless disease progression or evidence of peramplanel-related toxicity is observed. Subjects treated according to this combinatorial protocol will show substantially improved clinical benefits over SOC or other conventional anti-glioma therapy.

### Example IV

### Anti-Cancer Activity of AMPAR Antagonists in Combination with Levetiracetam Co-Treatment

In additional clinical examples, patients are treated concomitantly with an AMPAR antagonist such as peramplanel and Levetiracetam (Keppra), which has been shown to augment temozolomide efficacy (Bobustuc et al. 2010) and reduce aggression-related adverse events in patients taking perampanel (Kanemura et al. 2019; Kim et al. 2015). Patients are administered perampanel as described above along with 500 mg levetiracetam 1 hour prior to the first radiation session. Levetiracetam is administered twice a day, the second time being at night before bed. Within illustrative methods for treating GBM, patients receive 200-500 mg levetiracetam twice a day throughout the first 6-week treatment period, during the 1 month rest period, and while patients are taking maintenance Temodar. Patients continue to receive levetiracetam and perampanel unless disease progression or evidence of drug-related toxicity is observed. Subjects treated according to this combinatorial protocol will show substantially improved clinical benefits over S(X or other conventional anti-cancer therapy.

### Example V

### Anti-Cancer Activity of AMPAR Antagonists in Combination with NMDA Receptor Antagonist Co-Treatment

In other clinical protocols useful within the invention, patients are treated concomitantly with an N-methyl-D-aspartate (NMDA) receptor antagonist, such as memantine. Memantine has been reported to exert anti-cancer effects, possibly by abrogating constitutively active growth pathways in cancer (Stepulak et al. 2005; Maraka et al. 2019). Patients receive perampanel along with 5-20 mg memantine orally prior to the first radiation session. Within illustrative methods for treating GBM, perampanel and memantine are administered once a day throughout the first 6-week treatment period, during the 1 month rest period, and while patients are taking maintenance Temodar. Patients are maintained on memantine and perampanel unless disease progression or evidence of drug-related toxicity is observed. Subjects treated according to this combinatorial protocol will show substantially improved clinical benefits over SOC or other conventional anti-cancer therapy. In addition to memantine, ketamine, an NMDA-antagonist used in the setting of pharmacoresistant depression, may also be used. Ketamine is given at doses ranging from 5-500 mg/day and started prior to the first radiation session. Ketamine and its active metabolite hydroxynorketamine (Zanos et al. 2016) may provide anti-cancer benefits (Malsy et al. 2015), and will be a beneficial adjunct within the methods of the invention, for example to complement standard of care+perampanel treatments.

### Example VI

### Anti-Cancer Activity of AMPAR Antagonists in Combination with Riluzole/Troriluzole Co-Treatment

Additional clinical methods of the invention employ an AMPAR antagonist such as perampanel coordinately administered with Riluzole/troriluzole (see, e.g., Khan et al. 2019). Patients receive perampanel as above in coordinate treatment with 20-50 mg Riluzole/troriluzole orally prior to the first radiation session. Within illustrative methods for treating GBM, perampanel and Riluzole/troriluzole are administered once a day throughout the first 6-week treatment period, during the 1 month rest period, and while patients are taking maintenance Temodar. Patients are maintained on Riluzole/troriluzole and perampanel unless disease progression or evidence of drug-related toxicity is observed. Subjects treated according to this combinatorial protocol will show substantially improved clinical benefits over SOC or other conventional anti-cancer therapy.

### Example VII

### Anti-Cancer Activity of AMPAR Antagonists in Combination with CSF1R Inhibitors

Additional clinical methods of the invention employ an AMPAR antagonist such as perampanel coordinately administered with a colony stimulating factor I receptor (CSF1R) inhibitor such as PLX3397 (plexidartinib) or PLX5562 (see, e.g., Yan et al. 2017; Butowski et al. 2016). Patients are administered perampanel as above in coordinate treatment with 100-1000 mg PLX3397 orally prior to the first radiation session. Within illustrative methods for treating GBM, perampanel and PLX3397 are administered together or separately once a day throughout the first 6-week treatment period, during the 1 month rest period, and while patients are taking maintenance Temodar. Patients are maintained on PLX3397 and perampanel unless disease progression or evidence of drug-related toxicity is observed. Subjects treated according to this combinatorial protocol will show substantially improved clinical benefits over SOC or other conventional anti-cancer therapy. While CSF1R inhibition is reported to provide pre-clinical anti-cancer benefits results (Patwardhan et al. 2014; Yan et al. 2017; Quail et al. 2016), most pre-clinical models of different types of cancer demonstrate acquired resistance throughout this treatment (Patwardhan et al. 2014; Quail et al. 2016). In particular, for brain cancer, it has been shown that insulin-like growth factor 1 (IGF1) induces glioma rebound in CSF1R inhibitor-treated mice (Quail et al. 2016). Surprisingly. AMPA-glutamate antagonism according to the methods described here will negate oncogenic effects of IGF1, and AMPA-glutamate antagonism will complement with CSF1R inhibition to lower tumor burden in co-treated subjects. In more detailed aspects of the invention, it is noted that PLX3397 may inhibit PDGFRB signaling in cancer (Patwardhan et al. 2014), and that PDGFRB reportedly coordinates an anti-oxidant program in cancer through NRF2 transcription (Yang et al. 2018; Nanjaiah et al. 2019). On this basis, according to the teachings herein. PLX3397 will be beneficially combined with glutamate antagonists like memantine, within AMPAR antagonist methods of the invention, to bolster combined efficacy by suppressing an anti-oxidant program, e.g., in glioma cells.

### Example VIII

### Anti-Cancer Activity of AMPAR Antagonists in Combination with Anti-Malarial Drugs

Additional clinical methods of the invention employ an AMPAR antagonist such as perampanel coordinately administered with one or more anti-malarial drugs such as chloroquine, hydroxychloroquine, primaquine and mefloquine (see, e.g., Johnson et al. 2015; Liu et al. 2016; Maraka et al. 2019). In exemplary protocols, patients receive perampanel as above along with 250 mg mefloquine orally prior to the first radiation session. Within illustrative methods for treating GBM, Mefloquine is administered once every two days throughout the first 6-week treatment period, during the 1 month rest period, and while patients are taking maintenance Temodar. Patients are maintained on the anti-malarial and perampanel unless disease progression or evidence of drug-related toxicity is observed. Subjects treated according to this combinatorial protocol will show substantially improved clinical benefits over SOC or other conventional anti-cancer therapy.

### Example VIX

### Anti-Cancer Activity of AMPAR Antagonists in Combination with Metformin/Phenformin

Additional clinical methods of the invention employ an AMPAR antagonist such as perampanel coordinately administered with metformin (see, e.g., Benjamin et al. 2016; Maraka et al. 2019). Patients receive perampanel as above along with 500-2000 mg metformin orally prior to the first radiation session. In exemplary protocols for treating GBM, perampanel and metformin are administered once a day throughout the first 6-week treatment period, during the 1 month rest period, and while patients are taking maintenance Temodar. Patients are maintained on the metformin and perampanel unless disease progression or evidence of drug-related toxicity is observed. Subjects treated according to this combinatorial protocol will show substantially improved clinical benefits over SOC or other conventional anti-cancer therapy.

### Example X

### Anti-Cancer Activity of AMPAR Antagonists in Combination with PD-1 Inhibitor Treatment

Additional clinical methods of the invention employ an AMPAR antagonist such as perampanel coordinately administered with anti-cancer biologics including programmed cell death protein 1 (PD-1) inhibitors, such as pembrolizumab or nivolumab (see, e.g., Nghiem et al. 2016; Motzer et al. 2015). Resistance to PD-1 antagonism has been attributed to TNF-α production in the tumor microenvironment (Neubert et al. 2018). Since Ampa-glutamate antagonism has been shown to reduce TNF-α secretion in a model of intraventricular hemorrhage (Dohare et al. 2016), AMPAR antagonist treatment according to the invention will augment the efficacy of PD-1 inhibitors. In exemplary protocols, patients are administered perampanel as above, in conjunction with 1-3 mg/kg pembrolizumab/nivolumab intravenously prior to the first radiation session. Within illustrative methods for treating GBM, patients then receive pembrolizumab/nivolumab every 2 weeks throughout the first 6-week treatment period, during the 1 month rest period, and while patients are taking maintenance Temodar. Treatment is continued thusly unless disease progression or evidence of drug-related toxicity appears. Subjects treated according to this combinatorial protocol will show substantially improved clinical benefits over SOC or other conventional anti-cancer therapy.

### Example XI

### Anti-Cancer Activity of AMPAR Antagonists in Combination with PD-1 Inhibitor+CSF1R Inhibitor Treatment

Within more detailed examples, AMPAR antagonists such as perampanel are coordinately administered with PD-1 inhibitors, and also with CSF1R inhibitors. Notably, while PD-1 inhibitors reportedly exhibit robust efficacy in some patients, they appear to have little to no therapeutic effects in other patients. Recently, it has been suggested that CSF1 and TNF-α secretion by tumor cells may stanch the efficacy of PD-1 therapy (Neubert et al. 2018). As disclosed herein, patients be treated with a combination of AMPAR antagonists PD-1 inhibitors and CSF1R inhibitors (e.g., with perampanel, pembrolizumab, and PLX3397) will benefit by reduced CSF1 signaling in the tumor microenvironment, combined with ampa-glutamate antagonist repression of TNF signaling (see, e.g., Dohare et al. 2016), negating PD-1 inhibitor resistance to yield enhanced clinical benefits over SOC or other conventional anti-cancer therapy.

### Example XII

### Anti-Cancer Activity of AMPAR Antagonists in Combination with Clemastine Fumarate Co-Treatment

Additional clinical methods of the invention employ an AMPAR antagonist such as perampanel coordinately administered with clemastine fumarate (see, e.g., Döbbeling et al, 2013; Le Joncour et al. 2019). Patients are administered perampanel as above along with 0.5-2.68 mg clemastine fumarate orally prior to the first radiation session. In illustrative protocols for treating GBM, perampanel and clemastine are taken once a day throughout the first 6-week treatment period, during the 1 month rest period, and while patients are taking maintenance Temodar. Patients are maintained on the clemastine fumarate and perampanel unless disease progression or evidence of drug-related toxicity is observed. Subjects treated according to this combinatorial protocol will show substantially improved clinical benefits over SOC or other conventional anti-cancer therapy.

### Example XIII

### Anti-Cancer Activity of AMPAR Antagonists in Combination with SSRI Co-Treatment

Additional clinical methods of the invention employ an AMPAR antagonist such as perampanel coordinately administered with one or more selective serotonin reuptake inhibitors (SSRIs) (see, e.g., Sun et al. 2018; Huang et al. 2011; Lin et al. 2010; Liu et al, 2015; Yuan et al. 2018; Raabe & Gentile, 2008). In exemplary protocols for treating GBM, patients arc administered perampanel along with the SSRI(s) orally prior to the first radiation session, then once a day throughout the first 6-week treatment period, during the 1 month rest period, and while patients are taking maintenance Temodar. Patients are maintained on the SSRI and perampanel unless disease progression or evidence of drug-related toxicity is observed. Subjects treated according to this combinatorial protocol will show substantially improved clinical benefits over SOC or other conventional anti-cancer therapy.

### Example XIV

### Anti-Cancer Activity of AMPAR Antagonists in Combination with TCA Co-Treatment

Additional clinical methods of the invention employ an AMPAR antagonist such as perampanel coordinately administered with one or more tricyclic antidepressants (TCAs) (see, e.g., Jahchan et al. 2013; Jeon et al. 2011; Raabe & Gentile, 2008; Reynolds & Miller, 1988; Sernagor et al. 1989; Stoll et al. 2007). Patients receive perampanel along with the TCA(s) orally prior to the first radiation session. In exemplary protocols for treating GBM, the perampanel and TCA are then each administered once a day throughout the first 6-week treatment period, during the 1 month rest period, and while patients are taking maintenance Temodar. Patients are maintained on the TCA and perampanel unless disease progression or evidence of drug-related toxicity is observed. Subjects treated according to this combinatorial protocol will show substantially improved clinical benefits over SOC or other conventional anti-cancer therapy.

### Example XV

### Anti-Cancer Activity of AMPAR Antagonists in Combination with Ampakine Co-Treatment

Additional clinical methods of the invention employ an AMPAR antagonist such as perampanel coordinately administered with one or more positive allosteric AMPA receptor modulators (Ampakines). In exemplary protocols, patients are administered perampanen1 in combination with one or more ampakines, such as 2,3,6a,7,8,9-hexahydro-11H-1,4-dioxino[2,3-g]pyrrolo[2,1-b][1,3]benzoxazine-11-one (“CX614”) (see, e.g., Radin et al, 2018). Though ampakines are thought to augment AMPA-mediated currents in neurons, they have also been reported to induce AMPA receptor desensitization and down regulation via endocytosis and degradation after prolonged treatment (Jourdi et al. 2005), whereby they may serve as functional antagonists. Within illustrative methods for treating GBM, patients are administered perampanel along with CX614 or another amplakine orally prior to the first radiation session, then once a day throughout the first 6-week treatment period, during the 1 month rest period, and while patients are taking maintenance Temodar. Patients are maintained on the ampakine(s) and perampanel unless disease progression or evidence of drug-related toxicity is observed. Subjects treated according to this combinatorial protocol will show substantially improved clinical benefits over SOC or other conventional anti-cancer therapy.

### Example XVI

### Anti-Cancer Activity of AMPAR Antagonists in Combination with Cannabinoid Co-Treatment

Additional clinical methods of the invention employ an AMPAR antagonist such as perampanel coordinately administered with one or more cannabinoids, for example tetrahydrocannabinol (THC) and/or cannabidiol (CBD) (see, e.g., Scott et al. 2014; Marcu et al. 2010; Shrivastava et al. 2011). In exemplary protocols for treating GBM, patients are administered perampanel along with 100-600 mg CBD and 1-100 mg THC prior to the first radiation session, then once a day throughout the first 6-week treatment period, during the 1 month rest period, and while patients are taking maintenance Temodar. Patients are maintained on the cannabinoid and perampanel therapy unless disease progression or evidence of drug-related toxicity is observed. Subjects treated according to this combinatorial protocol will show substantially improved clinical benefits over SOC or other conventional anti-cancer therapy. For treatment of GBM, cannabinoids have been reported to potent effects on glioma stem cells (López-Valero et al. 2018). Considering that AMPA receptors are overexpressed on glioma stem cells (Oh et al. 2012), the combinatorial treatment methods and compositions described here will sensitize resistant tumor cells to the DNA-damaging effects of Temodar and radiation therapy (McLendon et al. 2006; Chen et al, 2012) and thereby enhance clinical benefits. Further, cannabinoids reportedly exert oncolytic effects through induction of harmful reactive oxygen species (Shrivastava et al. 2011; Nanjaiah et al. 2019), whereby the methods and compositions of the invention combining cannabinoids with glutamate antagonists will negate antioxidant defenses in cancer cells and enhance clinical benefits, particularly in glioma patients.

### Example XVI

### Anti-Cancer Activity of AMPAR Antagonists in Combination with Disulfiram Co-Treatment

Yet additional clinical methods of the invention employ an AMPAR antagonist such as perampanel coordinately administered with disulfiram (see, e.g., Lun et al. 2016; Triscott et al. 2012). Disulfiram targets cancer stem cells and reportedly inhibits MGMT to boost efficacy of Temodar (Paranjpe et al. 2014). Within the methods of the invention, both disulfiram and perampanel augment Temodar's efficacy and refine targeting of cancer cells, yielding surprisingly enhanced benefits for treating SOC treatment-resistant cancers. Within exemplary methods for treating GBM, patients are administered perampanel along with 50-500 mg disulfiram orally prior to the first radiation session, then once daily throughout the first 6-week treatment period, during the 1 month rest period, and while patients are taking maintenance Temodar. Patients are maintained on the perampanel and disulfiram unless disease progression or evidence of drug-related toxicity is observed. Subjects treated according to this combinatorial protocol will show substantially improved clinical benefits over SOC or other conventional anti-cancer therapy.

The instant description and examples are provided for illustration, and those skilled in the art will realize that the invention extends to additional embodiments and aspects following the teachings herein, and is therefore not limited except as by the appended claims.

### REFERENCES

1. Grossman, S. A., Ye. X., Chamberlain. M., Mikkelsen, T., Batchelor, T., Desideri, S., . . . Fine, H. A. (2009). Talampanel with standard radiation and temozolomide in patients with newly diagnosed glioblastoma; A multicenter phase II trial. Journal of Clinical Oncology, 27(25), 4155-4161.
2. Gidal, B. E., Laurenza, A., Hussein, Z., Yang, H., Fain, R., Edelstein, J., . . . Ferry, J. (2015). Perampanel efficacy and tolerability with enzyme-inducing AEDs in patients with epilepsy. Neurology, 84(19), 1972-1980.
3. Bobustuc. G. C., Baker, C. H., Limaye, A., Jenkins. W. D., Pearl, G., Avgeropoulos, N. G., & Konduri, S. D. (2010). Levetiracetam enhances p53-mediated MGMT inhibition and sensitizes glioblastoma cells to temozolomide. Neuro-Oncology, 12(9), 917-927.
4. Kanemura, H., Sano, F., & Aihara, M. (2019). Usefulness of perampanel with concomitant levetiracetam for patients with drug-resistant epilepsy. European Journal of Paediatric Neurology, 23(1), 197-203.
5. Kim, Y., Kim, T., Joo, J., Han, J. H., Kim, Y. J., Kim, I. A., . . . Kim, C. (2015). Survival benefit of levetiracetam in patients treated with concomitant chemoradiotherapy and adjuvant chemotherapy with temozolomide for glioblastoma multiforme. Cancer, 121(17), 2926-2932.
6. Stepulak. A., Sifringer, M., Rzeski, W., Endesfelder, S., Gratopp, A., Pohl, E. E., . . . Ikonomidou. C. (2005). NMDA antagonist inhibits the extracellular signal-regulated kinase pathway and suppresses cancer growth. Proceedings of the National Academy of Sciences of the United States of America, 102(43), 15605-15610.
7. Maraka, S., Groves, M. D., Mammoser, A. G., Melguizo-Gavilanes, I., Conrad. C. A., Tremont-Lukats. I. W., . . . Penas-Prado. M. (2019). Phase 1 lead-in to a phase 2 factorial study of temozolomide plus memantine, mefloquine, and metformin as postradiation adjuvant therapy for newly diagnosed glioblastoma. Cancer, 125(3), 424-433.
8. Zanos. P., Moaddel, R., Morris, P. J., Georgiou, P., Fischell, J., Elmer, G. I., . . . Gould, T. D. (2016). NMDAR inhibition-independent antidepressant actions of ketamine metabolites. Nature, 533(7604), 481-486.
9. Malsy, M., Gebhardt, K., Gruber, M., Wiese, C., Graf, B., & Bundscherer, A. (2015). Effects of ketamine, s-ketamine, and MK 801 on proliferation, apoptosis, and necrosis in pancreatic cancer cells. BMC Anesthesiology, 15(1), 111-118.
10. Khan. A. J., LaCava, S., Mehta, M., Schiff, D., Thandoni, A., Jhawar, S., . . . Chen, S. (2019). The glutamate release inhibitor riluzole increases DNA damage and enhances cytotoxicity in human glioma cells, in vitro and in vivo. Oncotarget, 10(29), 2824-2834.
11. Yan, D., Kowal, J., Akkari, L., Schuhmacher, A. J., Huse, J. T., West, B. L., & Joyce, J. A. (2017). Inhibition of colony stimulating factor-1 receptor abrogates microenvironment-mediated therapeutic resistance in gliomas. Oncogene, 36(43), 6049-6058.
12. Butowski. N., Colman. H., De Groot. J. F., Omuro, A. M., Nayak, L., Wen, P. Y., . . . Prados. M. (2016). Orally administered colony stimulating factor I receptor inhibitor PLX3397 in recurrent glioblastoma: An ivy foundation early phase clinical trials consortium phase II study. Neuro-Oncology, 18(4), 557-564.
13. Patwardhan, P. P., Surriga, O., Beckman, M. J., de Stanchina, E., Dematteo, R. P., Tap, W. D., & Schwartz, G. K. (2014). Sustained inhibition of receptor tyrosine kinases and macrophage depletion by PLX3397 and rapamycin as a potential new approach for the treatment of MPNSTs. Clinical Cancer Research, 20(12), 3146-3158.
14. Yan, D., Kowal, J., Akkari, L., Schuhmacher, A. J., Huse, J. T., West, B. L., & Joyce, J. A. (2017). Inhibition of colony stimulating factor-I receptor abrogates microenvironment-mediated therapeutic resistance in gliomas. Oncogene, 36(43), 6049-6058.
15. Quail. D. F., Bowman. R. L., Akkari. L., Quick. M. L., Schuhmacher, A. J., Huse, J. T., . . . Joyce. J. A. (2016). The tumor microenvironment underlies acquired resistance to CSF-1R inhibition in gliomas. Science, 352(6288).
16. Stepulak, A., Sifringer, M., Rzeski, W., Brocke, K., Gratopp, A., Pohl, E. E., . . . Ikonomidou, C. (2007). AMPA antagonists inhibit the extracellular signal regulated kinase pathway and suppress lung cancer growth. Cancer Biology & Therapy, 6(12), 1908.
17. Yang, Z., Yang, Y., Deng, Y., Chen, Y., Chen, X., Zhang, L., . . . Liu, B. (2018). Inhibition of PDGFR by CP-673451 induces apoptosis and increases cisplatin cytotoxicity in NSCLC cells via inhibiting the Nrf2-mediated defense mechanism. Toxicology Letters, 295, 88-98.
18. Nanjaiah, N. D., Ramaswamy. P., Goswami. K., Hurmath Fathima, K., & Borkotokey, M. (2019). Survival of glioblastoma cells in response to endogenous and exogenous oxidative challenges; Possible implication of NMDA receptor mediated regulation of redox homeostasis. Cell Biology International, 11193, 1-10.
19. Johnson, C. E., Hunt, D. K., Wiltshire. M., Herbert, T. P., Sampson, J. R., Errington, R. J., . . . Tee, A. R. (2015). Endoplasmic reticulum stress and cell death in mTORC1-overactive cells is induced by nelfinavir and enhanced by chloroquine. Molecular Oncology, 9(3), 675-688.
20. Liu, Y., Chen, S., Xue, R., Zhao, J., & Di, M. (2016). Mefloquine effectively targets gastric cancer cells through phosphatase-dependent inhibition of PI3K/Akt/mTOR signaling pathway. Biochemical and Biophysical Research Communications, 470(2), 350-355.
21. Benjamin. D., Colombi, M., Hindupur, S. K., Betz. C., Lane, H. A., El-Shemerly, M. Y. M., . . . Hall. M. N. (2016). Syrosingopine sensitizes cancer cells to killing by metformin. Science Advances, 2(12), e1601756-e1601756.
22. Nghiem, P. T., Bhatia. S., Lipson, E. J., Kudchadkar, R. R., Miller, N. J., Annamalai, L., . . . Cheever, M. A. (2016). PD-1 blockade with pembrolizumab in advanced merkel-cell carcinoma. The New England Journal of Medicine, 374(26), 2542-2552.
23. Motzer, R. J., Escudier. B., McDermott, D. F., George, S., Hammers, H. J., Srinivas, S., . . . CheckMate 025 Investigators. (2015). Nivolumab versus everolimus in advanced renal-cell carcinoma. The New England Journal of Medicine, 373(19), 1803-1813.
24. Neubert, N. J., Schmittnaegel, M., Bordry, N., Nassiri, S., Wald. N., Martignier, C., . . . Speiser, D. E. (2018). T cell-induced CSF1 promotes melanoma resistance to PD1 blockade. Science Translational Medicine, 10(436), 1-14.
25. Dohare, P., Zia, M. T., Ahmed, E., Ahmed, A., Yadala, V., Schober, A. L., . . . Ballabh, P. (2016). AMPA-kainate receptor inhibition promotes neurologic recovery in premature rabbits with intraventricular hemorrhage. J Neurosci, 36(11), 3363-3377.
26. Döbbeling, U., Waeckerle-Men, Y., Zabel, F., Graf, N., Kündig, T. M., & Johansen, P. (2013). The antihistamines clemastine and desloratadine inhibit STAT3 and c-myc activities and induce apoptosis in cutaneous T-cell lymphoma cell lines. Experimental Dermatology, 22(2), 119.
27. Le Joncour. V., Filppu. P., Hyvönen, M., Holopainen, M., Turunen, S. P., Sihto, H., . . . Laakkonen, P. (2019). Vulnerability of invasive glioblastoma cells to lysosomal membrane destabilization. EMBO Molecular Medicine, 11(6), e9034.
28. Sun, D., Zhu, L., Zhao, Y., Jiang, Y., Chen, L., Yu, Y., & Ouyang, L. (2018). Fluoxetine induces autophagic cell death via eEF2K-AMPK-mTOR-ULK complex axis in triple negative breast cancer. Cell Proliferation, 51(2), e12402.
29. Huang, J., Chang, H., Chou, C., Shu, S., Kuo, C., Tsai, J., . . . Jan, C. (2011). The mechanism of sertraline-induced [Ca2+]i rise in human PC3 prostate cancer cells: Sertraline and human prostate cancer cells. Basic & Clinical Pharmacology & Toxicology, 109(2), 103-110.
30. Lin. C., Robert, F., Sukarich, R., Michnick, S., & Pelletier, J. (2010). The antidepressant sertraline inhibits translation initiation by curtailing mammalian target of rapamycin signaling. Cancer Research, 70(8), 3199-3208.
31. Yuan, I., Horng, C., Chen, V. C., Chen, C., Chen, L., Hsu, T., & Tzang, B. (2018). Escitalopram oxalate inhibits proliferation and migration and induces apoptosis in non-small cell lung cancer cells. Oncology Letters, 15(3), 3376-3382.
32. Sun, D., Zhu. I., Zhao, Y., Jiang, Y., Chen, L., Yu, Y., & Ouyang, L. (2018). Fluoxetine induces autophagic cell death via eEF2K-AMPK-mTOR-ULK complex axis in triple negative breast cancer. Cell Proliferation, 51(2), e12402.
33. Liu, K., Yang, S., Lin, Y., Lin, J., Lee. Y., Wang, J., . . . Shen, S. (2015). Fluoxetine, an antidepressant, suppresses glioblastoma by evoking AMPAR-mediated calcium-dependent apoptosis. Oncotarget, 6(7), 5088-5101.
34. Raabe, R., & Gentile, L. (2008). Antidepressant interactions with the NMDA NR1-1b subunit. Journal of Biophysics, 2008, 474205-8.
35. Jahchan. N. S., Dudley. J. T., Mazur, P. K., Flores, N., Yang, D., Palmerton, A., . . . Sage, J. (2013). A drug repositioning approach identifies tricyclic antidepressants as inhibitors of small cell lung cancer and other neuroendocrine tumors. Cancer Discovery, 3(12), 1364-1377.
36. Jeon, S., Kim, Y. S., Kim. Y., Kim, S. H., Lim, Y., Lee, Y. H., & Shin, S. Y. (2011). The tricyclic antidepressant imipramine induces autophagic cell death in U-87MG glioma cells. Biochemical and Biophysical Research Communications, 413(2), 311-317.
37. Reynolds. I. J., & Miller. R. J. (1988). Tricyclic antidepressants block N-methyl-D-aspartate receptors: Similarities to the action of zinc. British Journal of Pharmacology, 95(1), 95-102.
38. Sernagor, E., Kuhn, D., Vyklicky, L., & Mayer, M. L. (1989). Open channel block of NMDA receptor responses evoked by tricyclic antidepressants. Neuron, 2(3), 1221-1227.
39. Stoll, L., Seguin, S., & Gentile, L. (2007). Tricyclic antidepressants, but not the selective serotonin reuptake inhibitor fluoxetine, bind to the S1S2 domain of AMPA receptors. Archives of Biochemistry and Biophysics, 458(2), 213-219.
40. Radin. D. P., Purcell. R., & Lippa, A. S. (2018). Oncolytic properties of ampakines in vitro. Anticancer Research, 38(1), 265-269.
41. Jourdi, H., Lu, X., Yanagihara, T., Lauterborn, J. C., Bi, X., Gall, C. M., & Baudry, M. (2005). Prolonged positive modulation of alpha-amino-3-hydroxy-5-methyl-4-isoxazolepropionic acid (AMPA) receptors induces calpain-mediated PSD-95/DIg/ZO-1 protein degradation and AMPA receptor down-regulation in cultured hippocampal slices. The Journal of Pharmacology and Experimental Therapeutics, 314(1), 16.
42. Scott, K. A., Dalgleish, A. G., & Liu, W. M. (2014). The combination of cannabidiol and Δ9-tetrahydrocannabinol enhances the anticancer effects of radiation in an orthotopic murine glioma model. Molecular Cancer Therapeutics, 13(12), 2955-2967.
43. Marcu. J. P., Christian. R. T., Lau, D., Zielinski, A. J., Horowitz, M. P., Lee, J., . . . McAllister. S. D. (2010). Cannabidiol enhances the inhibitory effects of Δ9-tetrahydrocannabinol on human glioblastoma cell proliferation and survival. Molecular Cancer Therapeutics, 9(1), 180.
44. Shrivastava, A., Kuzontkoski, P. M., Groopman, J. E., & Prasad, A. (2011). Cannabidiol induces programmed cell death in breast cancer cells by coordinating the cross-talk between apoptosis and autophagy. Molecular Cancer Therapeutics, 10(7), 1161-1172.
45. López-Valero, L., Saiz-Ladera, C., Torres. S., Hernandez-Tiedra, S., Garcia-Taboada, E., Rodriguez-Fornés. F., . . . Velasco. G. (2018). Targeting glioma initiating cells with A combined therapy of cannabinoids and temozolomide. Biochemical Pharmacology, 157, 266-274.
46. Oh, M. C., Kim, J. M., Safaee, M., Kaur, G., Sun, M. Z., Kaur, R., . . . Parsa, A. T. (2012). Overexpression of calcium-permeable glutamate receptors in glioblastoma derived brain tumor initiating cells. PloS One, 7(10), e47846-e47846.
47. McLendon, R. E., Hjelmeland, A. B., Dewhirst, M. W., Shi, Q., Rich, J. N., Wu, Q., . . . Hao, Y. (2006). Glioma stem cells promote radioresistance by preferential activation of the DNA damage response. Nature, 444(7120), 756-760.
48. Chen, J., Li. Y., Yu. T., McKay, R. M., Burns. D. K., Kernie, S. G., & Parada, L. F. (2012). A restricted cell population propagates glioblastoma growth after chemotherapy. Nature, 488(7412), 522-526.
49. Lun, X., Wells, J. C., Grinshtein, N., King. J. C., Hao, X., Dang, N., . . . Senger, D. L. (2016). Disulfiram when combined with copper enhances the therapeutic effects of temozolomide for the treatment of glioblastoma. Clinical Cancer Research, 22(15), 3860-3875.
50. Triscott, J., Lee. C., Hu. K., Fotovati, A., Berns, R., Pambid, M., . . . Dunn, S. E. (2012). Disulfiram, a drug widely used to control alcoholism, suppresses the self-renewal of glioblastoma and over-rides resistance to temozolomide. Oncotarget, 3(10), 1112-1123.
51. Paranjpe, A., Zhang, R., Ali-Osman, F., Bobustuc, G. C., & Srivenugopal, K. S. (2014). Disulfiram is a direct and potent inhibitor of human O6-methylguanine-DNA methyltransferase (MGMT) in brain tumor cells and mouse brain and markedly increases the alkylating DNA damage. Carcinogenesis, 35(3), 692-702.
52. Herner, A., Sauliunaite, D., Michalski. C. W., Erkan, M., De Oliveira, T., Abiatari, I., . . . Kleeff, J. (2011). Glutamate increases pancreatic cancer cell invasion and migration via AMPA receptor activation and Kras-MAPK signaling. Int J Cancer, 129(10), 2349-2359.
53. Ishiuchi. S., Tsuzuki, K., Yoshida. Y., Yamada, N., Hagimura, N., Okado, H., . . . Ozawa, S. (2002). Blockage of Ca(2+)-permeable AMPA receptors suppresses migration and induces apoptosis in human glioblastoma cells. Nat Med, 8(9), 971-978.
54. Ishiuchi, S., Yoshida. Y., Sugawara, K., Aihara, M., Ohtani, T., Watanabe, T., . . . Ozawa. S. (2007). Ca2+-permeable AMPA receptors regulate growth of human glioblastoma via Akt activation. J Neurosci, 27(30), 7987-8001.
55. von Roemeling, C. A., Radisky, D. C., Marlow, L. A., Cooper, S. J., Grebe, S. K., Anastasiadis. P. Z., . . . Copland. J. A. (2014). Neuronal pentraxin 2 supports clear cell renal cell carcinoma by activating the AMPA-selective glutamate receptor-4. Cancer Res, 74(17), 4796-4810.
56. Prasad, G., Sottero, T., Yang, X., Mueller, S., James, C. D., Weiss, W. A., . . . Haas-Kogan, D. A. (2011). Inhibition of PI3K/mTOR pathways in glioblastoma and implications for combination therapy with temozolomide. Neuro Oncol, 13(4), 384-392.
57. Piao. Y., Lu. L., & de Groot. J. (2009). AMPA receptors promote perivascular glioma invasion via beta 1 integrin-dependent adhesion to the extracellular matrix. Neuro Oncol. 11(3), 260-273.
58. Hu. H., Takano, N., Xiang, L., Gilkes, D. M., Luo, W., & Semenza, G. L. (2014). Hypoxia-inducible factors enhance glutamate signaling in cancer cells. Oncotarget, 5(19), 8853-8868.
59. Takano. T., Lin, J. H., Arcuino, G., Gao, Q., Yang, J., & Nedergaard, M. (2001). Glutamate release promotes growth of malignant gliomas. Nat Med, 7(9), 1010-1015.
60. Rzeski. W., Turski, L., & Ikonomidou, C. (2001). Glutamate antagonists limit tumor growth. Proc Natl Acad Sci USA, 98(11), 6372-6377.
61. Chen. C., Matt, L., . . . Hell, J. W., & Rogawski, M. A. (2014). Perampanel inhibition of AMPA receptor currents in cultured hippocampal neurons. PloS One, 9(9), e108021.
62. Patsalos, P N. (2015). The clinical pharmacology profile of the new antiepileptic drug perampanel: A novel noncompetitive AMPA receptor antagonist. Epilepsia, 56(1), 12-27.
63. Biancur, D. E., Paulo. J. A., Malachowska, B., Maria Quiles Del Rey, Sousa, C. M., Wang, X., . . . Kimmelman, A. C. (2017). Compensatory metabolic networks in pancreatic cancers upon perturbation of glutamine metabolism. Nature Communications, 8, 15965.

What is claimed:

1. A method for treating an AMPA Receptor (AMPAR) positive cancer in a mammalian subject comprising administering an anti-cancer effective amount of an AMPAR antagonist compound to said subject.

2. The method of claim 1, wherein the AMPAR antagonist compound is an allosteric AMPAR antagonist compound.

3. The method of claim 1, wherein the AMPAR antagonist compound is a Perampanel (PMP) compound.

4. The method of claim 3, wherein the PMP compound is an anti-cancer effective analog or derivative of PMP.

5. The method of claim 1, wherein the AMPA Receptor (AMPAR) positive cancer is selected from an AMPA Receptor (AMPAR) positive brain cancer, lung cancer, prostate cancer, breast cancer; skin cancer, liver cancer, thyroid cancer, esophageal cancer, sarcoma, colorectal cancer, bladder cancer, gall bladder cancer, stomach cancer, renal cancer, ovarian cancer, uterine cancer, cervical cancer, non-Hodgkin's lymphoma; acute myelogenous leukemia (AML), acute lymphocytic leukemia, chronic lymphocytic leukemia (CLL), myeloma, mesothelioma, pancreatic cancer, Hodgkin's disease, testicular cancer, Waldenstrom's disease, head/neck cancer, tongue cancer, or viral-induced cancer.

6. The method of claim 1, wherein the AMPA Receptor (AMPAR) positive cancer is selected from an AMPA Receptor (AMPAR) positive glioblastoma (GBM), or cancer of the breast, pancreas, lung or kidney.

7. The method of claim 1, wherein the AMPA Receptor (AMPAR) positive cancer is an AMPA Receptor (AMPAR) positive glioblastoma (GBM).

8. The method of claim 1, wherein the AMPAR positive cancer is a glioblastoma (GBM).

9. The method of claim 1, wherein the AMPAR antagonist compound is Perampanel (PMP) formulated in an aqueous carrier for parenteral or intravenous administration.

10. The method of claim 1, wherein the AMPAR antagonist compound is Perampanel (PMP) formulated in an oral dosage form.

11. The method of claim 1, wherein the AMPAR antagonist compound mediates a greater than 20% increase in cancer free survival for treated subjects compared to control subjects.

12. The method of claim 1, wherein the AMPAR antagonist compound mediates a 20-50% or greater increase in cancer free survival for subjects compared to control subjects.

13. The method of claim 1, wherein the AMPAR antagonist compound mediates at least a 20% decrease in average size or number of primary tumors or metastases in treated subjects compared to control subjects.

14. The method of claim 1, wherein the AMPAR antagonist compound mediates a 20-50% or larger decrease in average size or number of primary tumors or metastases in treated subjects compared to control subjects.

15. The method of claim 1, wherein the AMPAR antagonist compound is Perampanel (PMP) or an anti-cancer effective prodrug, metabolite, analog or derivative of PMP.

16. The method of claim 1, further comprising coordinately administering a secondary anti-cancer agent or therapy to the subject.

17. The method of claim 16, wherein the secondary anti-cancer agent or therapy is selected from tubulin depolymerizing agents, DNA damaging agents, inhibitors of DNA synthesis, anti-metabolics, anti-angiogenic agents, vascular disrupting agents (VDAs), anti-cancer antibodies, endocrine cancer therapies, immuno-modulators, histone deacetylase inhibitors, inhibitors of signal transduction, inhibitors of heat shock proteins, retinoids, growth factors, growth factor receptor modulators, anti-mitotic compounds, anti-inflammatory drugs, and cell cycle regulators.

18. The method of claim 16, wherein the secondary anti-cancer agent or therapy is selected from anti-cancer chemotherapy, surgery and radiation.

19. The method of claim 16, further comprising coordinately administering one or more secondary anti-cancer, chemotherapeutic agent(s) selected from azacitidine, bevacizumab, bortezomib, capecitabine, cetuximab, clofarabine, dasatinib, decitabine, docetaxel, emend, erlotinib hydrochloride, exemestane, fulvestrant, gefitinib, gemcitabine hydrochloride, imatinib mesylate, imiquimod, lenalidomide, letrozole, nelarabine, oxaliplatin, paclitaxel, docetaxel, palifermin, panitumumab, pegaspargase, pemetrexed disodium, rituximab, sorafenib tosylate, sunitinib malate, tamoxifen citrate, targretin, temozolomide, thalidomide, and/or topotecan hydrochloride.

20. The method of claim 16, further comprising coordinately administering one or more secondary anti-cancer agents selected from an interleukin, interferon, filgrasten, G-CSF, epoetin alfa, erythropoietin, and/or an anti-cancer antibody or antibody fragment.

21. The method of claim 16, wherein the AMPAR antagonist compound is Perampanel (PMP) or an anti-cancer effective prodrug, metabolite, analog or derivative of PMP, and the secondary anti-cancer agent is selected from temozolomide (TMZ), a transcription inhibitor, a telomere disrupting agent, an inhibitor of a gene splicing protein, an indoleamine 2, 3, dioxegenase (IDO) inhibitor, a lapatinib ditosylate enzyme blocker, and an anti-cancer antibody or antibody fragment.

22. The method of claim 16, wherein the AMPAR antagonist compound is Perampanel (PMP) or an anti-cancer effective prodrug, metabolite, analog or derivative of PMP, and the secondary anti-cancer therapy employs tumor treating fields or radiation.

23. A pharmaceutical composition comprising an anti-cancer effective amount of an AMPAR antagonist compound and a secondary anti-cancer agent selected from a tubulin depolymerizing agent, a DNA damaging agents, an inhibitor of DNA synthesis, an anti-metabolic drug, an anti-angiogenic agent, a vascular disrupting agent (VDAs), and anti-cancer antibody or antibody fragment, an anti-cancer cytokine, an anti-cancer hormone, a histone deacetylase inhibitor, a retinoid, a growth factor, a growth factor receptor modulator, an anti-mitotic compound, or a cell cycle regulator compound.

24. The pharmaceutical composition of claim 23, wherein the secondary anti-cancer agent is selected from temozolomide (TMZ), a transcription inhibitor, a telomere disrupting agent, an inhibitor of a gene splicing protein, an indoleamine 2, 3, dioxegenase (IDO) inhibitor, a lapatinib ditosylate enzyme blocker, and an anti-cancer antibody or antibody fragment.

25. The method of claim 1, further comprising coordinately administering a secondary therapeutic agent or method to the subject selected from NMDA antagonists; anti PD-1/PDL-1 therapy; CSF1R inhibitors; cannabinoid drugs; anti-malarials; Riluzole/troriluzole treatment; antihistamines; biguanides; anti-cancer biologics; SSRIs; TCAs; Ampakines; levetiracetam, or a combination thereof.

26. The pharmaceutical composition of claim 23 comprising an anti-cancer effective amount of an AMPAR antagonist compound and a secondary therapeutic agent selected from NMDA antagonists; anti PD-1/PDL-1 drugs; CSF1R inhibitors; cannabinoid drugs; anti-malarials; Riluzole/troriluzole; antihistamines; biguanides; anti-cancer biologics; SSRIs; TCAs; Ampakines; levetiracetam, or a combination thereof.
